(19) United States
(12) Patent Application Publication
(10) Pub. No.: US 2025/0288234 A1
(43) Pub. Date: Sep. 18, 2025

(54) SYSTEMS AND METHODS FOR CAPACITANCE-BASED MEASUREMENT OF BODILY FLUIDS

(71) Applicant: UNIVERSITY OF FLORIDA RESEARCH FOUNDATION, INCORPORATED, Gainesville, FL (US)

(72) Inventors: Anatoliy KORZHUK, Gainesville, FL (US); Khaled DEEB, Gainesville, FL (US); Joseph DEEB, Gainesville, FL (US)

(21) Appl. No.: 19/072,713

(22) Filed: Mar. 6, 2025

Related U.S. Application Data
(60) Provisional application No. 63/564,072, filed on Mar. 12, 2024.

(51) Int. Cl. A61B 5/20; A61B 5/00; G01F 23/263; G16H 30/20

(52) U.S. Cl. CPC A61B 5/208; A61B 5/0004; G01F 23/266; G16H 30/20

(57) ABSTRACT

Various embodiments of the present disclosure provide systems and methods for capacitance-based measurement of bodily fluids. An example system may include a fluid collection reservoir configured to collect one or more bodily fluids of an individual, a first capacitance sensor configured to output a first capacitance value indicative of a first fluid level of the one or more bodily fluids, a second capacitance sensor configured to output a second capacitance value indicative of a second fluid level of the one or more bodily fluids, and one or more processors configured to receive the first capacitance value and the second capacitance value and determine a third fluid level of the one or more bodily fluids based at least in part on the first fluid level and the second fluid level, where the third fluid level is indicative of one or more health metrics for the individual.

### CROSS-REFERENCE TO RELATED APPLICATIONS

This application claims priority to U.S. Provisional Application No. 63/564,072, filed on Mar. 12, 2024, the contents of which are hereby incorporated by reference in their entirety.

### TECHNICAL FIELD

The present application relates to the technical field of healthcare. In particular, the invention relates to systems and methods for capacitance-based measurement of bodily fluids.

### BACKGROUND

Various devices may be utilized to collect fluids for different applications. For example, fluid collection devices may be utilized for healthcare applications, industrial applications, automotive applications, agricultural applications, building system applications, and/or the like. In some examples, a fluid collection device may include one or more fluid level markings indicative of a volume of a fluid in the fluid collection device. Such fluid level markings may be utilized by one or more individuals to manually determine a fluid level within the fluid collection device. However, the manual monitoring of a fluid level within a fluid collection device may present various challenges, particularly in environments where the potential for human error poses various safety hazards, or in time-constrained environments where workforce resources are limited.

### BRIEF SUMMARY

In accordance with one or more examples described herein, a fluid measurement device is provided. The fluid measurement device may include a fluid collection reservoir and one or more capacitance sensors, such as metallic rod or flexible tape printed surface board, positioned in the fluid collection reservoir. The one or more capacitance sensors may be configured to output one or more signals to one or more processors, which may be located in a base of the fluid measurement device. The one or more processors may receive the one or more signals and may determine information based on the one or more signals.

For example, the one or more processors may determine a tilt angle of the fluid measurement device and/or one or more fluid levels of fluid in the fluid collection reservoir. In some examples, the one or more fluid levels may be determined by the one or more processors based on the tilt angle.

In some examples, the one or more processors may determine whether one or more measurement accuracy criteria are satisfied (e.g., based on the one or more signals received from the one or more capacitance sensors). In such examples, the one or more processors may determine one or more measurement timings (e.g., for determining one or more fluid levels) based on whether the one or more measurement accuracy criteria are satisfied. Accordingly, the one or more processors may avoid determining, generating, or outputting one or more fluid level values if the fluid measurement device has been agitated, if the fluid measurement device is tilted, and/or if an error has occurred (e.g., if a capacitance sensor fails to output a signal), which may improve measurement accuracy.

In some examples, the fluid measurement device may communicate one or more fluid level values to a medical record or to a computing entity that stores a medical record. Such communications may enable health data for one or more individuals to be recorded more efficiently when compared to conventional techniques that involve manual recordation of data. In some examples, the fluid measurement device may communicate the one or more fluid level values to the medical record via a specific type of message that protects the identity of a patient (e.g., names and social security numbers of patients are not included in the message). For example, the fluid measurement device may communicate the one or more fluid level values to the medical record using a health level 7 (HL7) message. In such examples, the fluid measurement device may receive a patient identifier (e.g., other than a name or social security number for the patient) and may link the patient identifier to the one or more fluid level values. In some examples, a healthcare provider may then retrieve the data and prescribe a treatment and/or medication to the patient based on the data. The patient may then perform one or more actions (e.g., adhere to a treatment plan), which may result in an improved health outcome for the patient.

In one embodiment, a system includes a fluid collection reservoir configured to collect one or more bodily fluids associated with an individual; a first capacitance sensor positioned in the fluid collection reservoir and configured to output a first capacitance value indicative of a first fluid level of the one or more bodily fluids at a first location within the fluid collection reservoir; a second capacitance sensor positioned in the fluid collection reservoir and configured to output a second capacitance value indicative of a second fluid level of the one or more bodily fluids at a second location within the fluid collection reservoir; and one or more processors in communication with the first capacitance sensor and the second capacitance sensor, the one or more processors configured to receive the first capacitance value and the second capacitance value and determine a third fluid level of the one or more bodily fluids based at least in part on the first fluid level and the second fluid level, wherein the third fluid level is indicative of one or more health metrics for the individual.

In some examples, the one or more processors are further configured to: determine a tilt angle of the fluid collection reservoir based at least in part on a comparison of the first capacitance value and the second capacitance value, wherein the third fluid level is based at least in part on the tilt angle. In some examples, the one or more processors are further configured to: determine whether one or more measurement accuracy criteria are satisfied based at least in part on the first capacitance value and the second capacitance value; and determine a measurement timing for determining the third fluid level based at least in part on whether the one or more measurement accuracy criteria are satisfied.

In some examples, the one or more measurement accuracy criteria comprise one or more of (i) a tilt level criteria and (ii) an agitation criteria. In some examples, the one or more processors are further configured to: generate a message comprising at least an indication of the third fluid level; and cause transmission of the message to an electronic medical record for the individual. In some examples, the one or more processors are further configured to receive an identification number for the individual and the electronic medical record for the individual is identified based at least in part on the identification number for the individual.

In some examples, the one or more processors configured to generate the message comprising at least the indication of the third fluid level comprise one or more processors configured to format the message according to a health level seven (HL7) messaging standard. In some examples, the one or more processors configured to cause transmission of the message comprise one or more processors configured to encode the message according to a security protocol consistent with an electronic medical record system associated with the electronic medical record for the individual.

In one embodiment, a method includes outputting, by a first capacitance sensor disposed in a fluid collection reservoir, a first capacitance value indicative of a first fluid level of one or more bodily fluids at a first location within the fluid collection reservoir, the one or more bodily fluids associated with an individual; outputting, by a second capacitance sensor disposed in the fluid collection reservoir, a second capacitance value indicative of a second fluid level of the one or more bodily fluids at a second location within the fluid collection reservoir; receiving, by one or more processors, the first capacitance value and the second capacitance value; and determining, by the one or more processors, a third fluid level of the one or more bodily fluids based at least in part on the first fluid level and the second fluid level, wherein the third fluid level is indicative of one or more health metrics for the individual.

In some examples, the method further includes determining, by the one or more processors, a tilt angle of the fluid collection reservoir based at least in part on a comparison of the first capacitance value and the second capacitance value, wherein the third fluid level is based at least in part on the tilt angle. In some examples, the method further includes determining, by the one or more processors, whether one or more measurement accuracy criteria are satisfied based at least in part on the first capacitance value and the second capacitance value; and determining, by the one or more processors, a measurement timing for determining the third fluid level based at least in part on whether the one or more measurement accuracy criteria are satisfied.

In some examples, the one or more measurement accuracy criteria comprise one or more of (i) a tilt level criteria and (ii) an agitation criteria. In some examples, the method further includes generating, by the one or more processors, a message comprising at least an indication of the third fluid level; and causing, by the one or more processors, transmission of the message to an electronic medical record for the individual. In some examples, the method further includes receiving, by the one or more processors, an identification number for the individual, wherein the electronic medical record for the individual is identified based at least in part on the identification number for the individual.

In some examples, the method further includes formatting, by the one or more processors, the message according to a health level seven (HL7) messaging standard. In some examples, the method further includes encoding, by the one or more processors, the message according to a security protocol consistent with an electronic medical record system associated with the electronic medical record for the individual.

In one embodiment, an apparatus includes at least one processor; and at least one memory storing instructions that, when executed by the at least one processor, cause the apparatus to: output, by a first capacitance sensor disposed in a fluid collection reservoir, a first capacitance value indicative of a first fluid level of one or more bodily fluids at a first location within the fluid collection reservoir, the one or more bodily fluids associated with an individual; output, by a second capacitance sensor disposed in the fluid collection reservoir, a second capacitance value indicative of a second fluid level of the one or more bodily fluids at a second location within the fluid collection reservoir; receive, by the at least one processor, the first capacitance value and the second capacitance value; and determine, by the at least one processor, a third fluid level of the one or more bodily fluids based at least in part on the first fluid level and the second fluid level, wherein the third fluid level is indicative of one or more health metrics for the individual.

In some examples, the instructions, when executed by the at least one processor further cause the apparatus to: determine, by the at least one processor, a tilt angle of the fluid collection reservoir based at least in part on a comparison of the first capacitance value and the second capacitance value, wherein the third fluid level is based at least in part on the tilt angle. In some examples, the instructions, when executed by the at least one processor further cause the apparatus to: determine, by the at least one processor, whether one or more measurement accuracy criteria are satisfied based at least in part on the first capacitance value and the second capacitance value; and determine, by the at least one processor, a measurement timing for determining the third fluid level based at least in part on whether the one or more measurement accuracy criteria are satisfied.

In some examples, the one or more measurement accuracy criteria comprise one or more of (i) a tilt level criteria and (ii) an agitation criteria. In some examples, the instructions, when executed by the at least one processor further cause the apparatus to: generate, by the at least one processor, a message comprising at least an indication of the third fluid level; and cause, by the at least one processor, transmission of the message to an electronic medical record for the individual. In some examples, the instructions, when executed by the at least one processor further cause the apparatus to: receive, by the at least one processor, an identification number for the individual, wherein the electronic medical record for the individual is identified based at least in part on the identification number for the individual.

### BRIEF DESCRIPTION OF THE DRAWINGS

Reference will now be made to the accompanying drawings, which are not necessarily drawn to scale, and wherein:

FIG. 1 illustrates an example computing system in accordance with some embodiments of the present disclosure.

FIG. 2 is a schematic diagram showing a system computing architecture in accordance with some embodiments discussed herein.

FIG. 3 illustrates a data flow diagram in accordance with some embodiments discussed herein.

FIG. 4 illustrates an operational example of a urinal in accordance with some embodiments discussed herein.

FIG. 5 illustrates an operational example of a urinal in accordance with some embodiments discussed herein.

FIG. 6 illustrates an operational example of an electronics assembly in accordance with some embodiments discussed herein.

FIG. 7 illustrates an operational example of a wall cannister in accordance with some embodiments discussed herein.

FIG. 8 illustrates an operational example of a wall cannister in accordance with some embodiments discussed herein.

FIG. 9 illustrates an operational example of an electronics assembly in accordance with some embodiments discussed herein.

FIG. 10 illustrates an operational example of a chest tube cannister in accordance with some embodiments discussed herein.

FIG. 11 illustrates an operational example of a chest tube cannister in accordance with some embodiments discussed herein.

FIG. 12 illustrates an operational example of an electronics assembly in accordance with some embodiments discussed herein.

FIG. 13 provides an example flowchart in accordance with some embodiments discussed herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout.

### I. COMPUTER PROGRAM PRODUCTS, METHODS, AND COMPUTING ENTITIES

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established, or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In some embodiments, a non-volatile computer-readable storage medium may include a floppy disk, flexible disk, hard disk, solid-state storage (SSS) (e.g., a solid-state drive (SSD), solid state card (SSC), solid state module (SSM), enterprise flash drive, magnetic tape, or any other non-transitory magnetic medium, and/or the like). A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In some embodiments, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for, or used in addition to, the computer-readable storage media described above.

As should be appreciated, various embodiments of the present disclosure may also be implemented as methods, apparatuses, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises a combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments may produce specifically configured machines performing the steps or operations specified in the block diagrams and flowchart illustrations. Accordingly, the block diagrams and flowchart illustrations support various combinations of embodiments for performing the specified instructions, operations, or steps.

### II. EXAMPLE SYSTEM FRAMEWORK

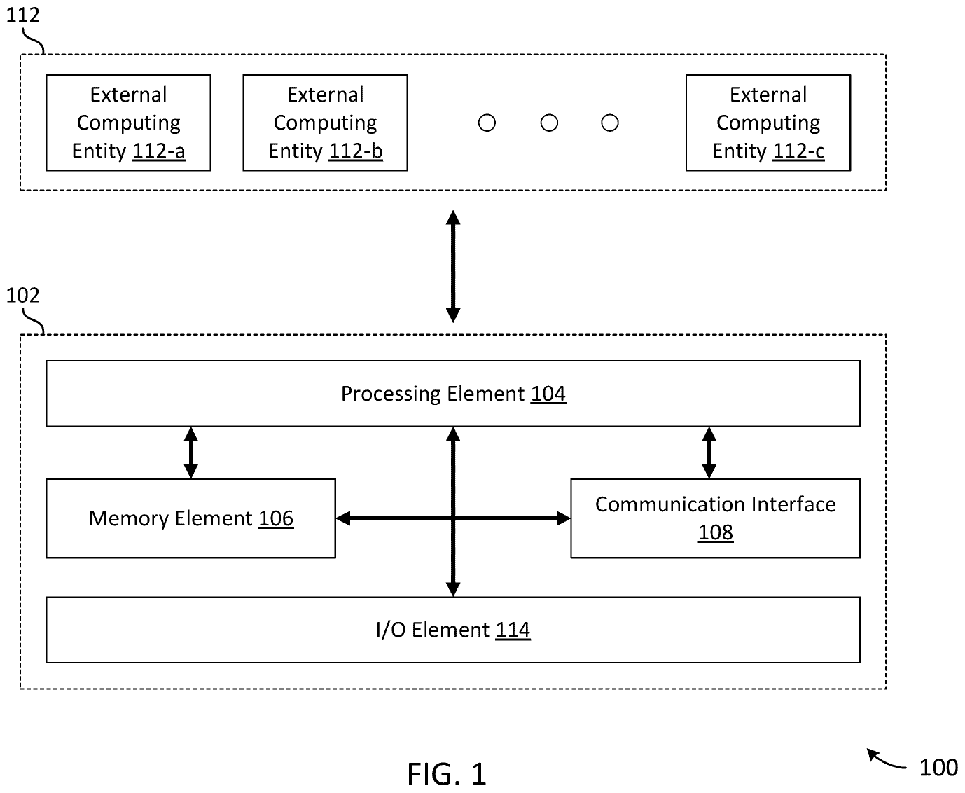

FIG. 1 illustrates an example computing system 100 in accordance with one or more embodiments of the present disclosure. The computing system 100 may include a computing entity 102 and/or one or more external computing entities 112 (e.g., external computing entity 112-a, external computing entity 112-b, external computing entity 112-c) communicatively coupled to the computing entity 102 using one or more wired and/or wireless communication techniques. The computing entity 102 may be specially configured to perform one or more steps/operations of one or more techniques described herein. In some embodiments, the computing entity 102 may include and/or be in association with one or more mobile device(s), desktop computer(s), laptop(s), server(s), cloud computing platform(s), and/or the like. In some example embodiments, the computing entity 102 may be configured to receive and/or transmit information, such as one or more datasets, data objects, and/or the like from and/or to the external computing entities 112 to perform one or more steps/operations as described herein (e.g., steps/operations for capacitance-based measurement of bodily fluids).

The computing entity 102, for example, may include and/or be associated with one or more entities that may be configured to receive, transmit, store, and/or manage information, such as information indicative of one or more fluid levels in a fluid collection reservoir. For example, a fluid measurement device (e.g., a fluid collection device) as described herein may include one or more computing entities 102, which may be configured to receive, determine, generate, and/or transmit information, such as information indicative of one or more fluid levels. Additionally, or alternatively, the computing entity 102 may receive, determine, generate, and/or transmit various other types of information, such as timing information (e.g., a current date and time, a date and time of one or more fluid levels), one or more health metrics, one or more tilt angles of a fluid collection reservoir, identification information for an individual, and/or the like). In some examples, the computing entity 102 may display information via a user interface of the computing entity 102. Additionally, or alternatively, the computing entity 102 may display information via a user interface of an external computing entity 112. In some examples, the computing entity 102 may communicate information to an electronic medical record, which may be stored or managed by one or more external computing entities 112.

The external computing entities 112, for example, may include and/or be associated with one or more entities that may be configured to receive, transmit, store, and/or manage information, such as information indicative of one or more fluid levels, timing information (e.g., a current date and time, a date and time of one or more fluid levels), one or more health metrics, one or more tilt angles of a fluid collection reservoir, identification information for an individual, and/or the like. The external computing entities 112, for example, may be associated with one or more data repositories, cloud platforms, compute nodes, organizations, and/or the like, which may be individually and/or collectively leveraged to obtain and aggregate data for an individual.

The computing entity 102 may include, or be in communication with, one or more processing elements 104 (also referred to as processors, processing circuitry, digital circuitry, and/or similar terms used herein interchangeably) that communicate with other elements within the computing entity 102 via a bus, for example. As will be understood, the computing entity 102 may be embodied in a number of different ways. The computing entity 102 may be configured for a particular use or configured to execute instructions stored in volatile or non-volatile media or otherwise accessible to the processing element 104. As such, whether configured by hardware or computer program products, or by a combination thereof, the processing element 104 may be capable of performing steps or operations according to embodiments of the present disclosure when configured accordingly.

In one embodiment, the computing entity 102 may further include, or be in communication with, one or more memory elements 106. The memory element 106 may be used to store at least portions of the databases, database instances, database management systems, data, applications, programs, program modules, scripts, source code, object code, byte code, compiled code, interpreted code, machine code, executable instructions, and/or the like being executed by, for example, the processing element 104. Thus, the databases, database instances, database management systems, data, information, applications, programs, program modules, scripts, source code, object code, byte code, compiled code, interpreted code, machine code, executable instructions, and/or the like, may be used to control certain aspects of the operation of the computing entity 102 with the assistance of the processing element 104.

As indicated, in one embodiment, the computing entity 102 may also include one or more communication interfaces 108 for communicating with various computing entities, e.g., external computing entities 112, such as by communicating data, content, information, and/or similar terms used herein interchangeably that may be transmitted, received, operated on, processed, displayed, stored, and/or the like.

The computing system 100 may include one or more input/output (I/O) element(s) 114 for communicating with one or more users. An I/O element 114, for example, may include one or more user interfaces for providing and/or receiving information from one or more users of the computing system 100. The I/O element 114 may include one or more tactile interfaces (e.g., keypads, touch screens, etc.), one or more audio interfaces (e.g., microphones, speakers, etc.), visual interfaces (e.g., display devices, etc.), and/or the like. The I/O element 114 may be configured to receive user input through one or more of the user interfaces from a user of the computing system 100 and provide data to a user through the user interfaces.

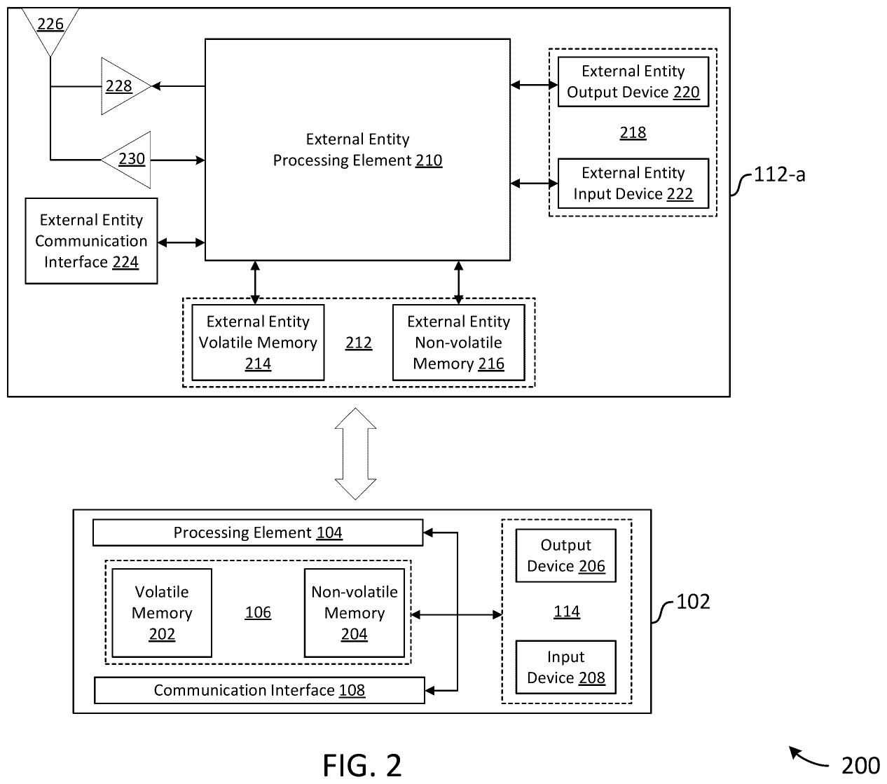

FIG. 2 is a schematic diagram showing a system computing architecture 200 in accordance with some embodiments discussed herein. In some embodiments, the system computing architecture 200 may include the computing entity 102 and/or the external computing entity 112-a of the computing system 100. The computing entity 102 and/or the external computing entity 112-a may include a computing apparatus, a computing device, and/or any form of computing entity configured to execute instructions stored on a computer-readable storage medium to perform certain steps or operations.

The computing entity 102 may include a processing element 104, a memory element 106, a communication interface 108, and/or one or more I/O elements 114 that communicate within the computing entity 102 via internal communication circuitry, such as a communication bus and/or the like.

The processing element 104 may be embodied as one or more complex programmable logic devices (CPLDs), microprocessors, multi-core processors, coprocessing entities, application-specific instruction-set processors (ASIPs), microcontrollers, and/or controllers. Further, the processing element 104 may be embodied as one or more other processing devices or circuitry including, for example, a processor, one or more processors, various processing devices, and/or the like. The term circuitry may refer to an entirely hardware embodiment or a combination of hardware and computer program products. Thus, the processing element 104 may be embodied as integrated circuits, application specific integrated circuits (ASICs), field programmable gate arrays (FPGAs), programmable logic arrays (PLAs), hardware accelerators, digital circuitry, and/or the like.

The memory element 106 may include volatile memory 202 and/or non-volatile memory 204. The memory element 106, for example, may include volatile memory 202 (also referred to as volatile storage media, memory storage, memory circuitry, and/or similar terms used herein interchangeably). In one embodiment, a volatile memory 202 may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for, or used in addition to, the computer-readable storage media described above.

The memory element 106 may include non-volatile memory 204 (also referred to as non-volatile storage, memory, memory storage, memory circuitry, and/or similar terms used herein interchangeably). In one embodiment, the non-volatile memory 204 may include one or more non-volatile storage or memory media, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

In one embodiment, a non-volatile memory 204 may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile memory 204 may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile memory 204 may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

As will be recognized, the non-volatile memory 204 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

The memory element 106 may include a non-transitory computer-readable storage medium for implementing one or more aspects of the present disclosure including as a computer-implemented method configured to perform one or more steps/operations described herein. For example, the non-transitory computer-readable storage medium may include instructions that when executed by a computer (e.g., processing element 104), cause the computer to perform one or more steps/operations of the present disclosure. For instance, the memory element 106 may store instructions that, when executed by the processing element 104, configure the computing entity 102 to perform one or more steps/operations described herein.

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language, such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established, or fixed) or dynamic (e.g., created or modified at the time of execution).

The computing entity 102 may be embodied by a computer program product which includes non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media such as the volatile memory 202 and/or the non-volatile memory 204.

The computing entity 102 may include one or more I/O elements 114. The I/O elements 114 may include one or more output devices 206 and/or one or more input devices 208 for providing and/or receiving information with a user, respectively. The output devices 206 may include one or more sensory output devices, such as one or more tactile output devices (e.g., vibration devices such as direct current motors, and/or the like), one or more visual output devices (e.g., liquid crystal displays, LEDs, and/or the like), one or more audio output devices (e.g., speakers, and/or the like), and/or the like. The input devices 208 may include one or more sensory input devices, such as one or more tactile input devices (e.g., touch sensitive displays, push buttons, and/or the like), one or more audio input devices (e.g., microphones, and/or the like), and/or the like.

In addition, or alternatively, the computing entity 102 may communicate, via a communication interface 108, with one or more external computing entities such as the external computing entity 112-a. The communication interface 108 may be compatible with one or more wired and/or wireless communication protocols.

For example, such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. In addition, or alternatively, the computing entity 102 may be configured to communicate via wireless external communication using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.9 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

The external computing entity 112-a may include an external entity processing element 210, an external entity memory element 212, an external entity communication interface 224, and/or one or more external entity I/O elements 218 that communicate within the external computing entity 112-a via internal communication circuitry, such as a communication bus and/or the like.

The external entity processing element 210 may include one or more processing devices, processors, and/or any other device, circuitry, and/or the like described with reference to the processing element 104. The external entity memory element 212 may include one or more memory devices, media, and/or the like described with reference to the memory element 106. The external entity memory element 212, for example, may include at least one external entity volatile memory 214 and/or external entity non-volatile memory 216. The external entity communication interface 224 may include one or more wired and/or wireless communication interfaces as described with reference to communication interface 108.

In some embodiments, the external entity communication interface 224 may be supported by one or more radio circuitry. For instance, the external computing entity 112-a may include an antenna 226, a transmitter 228 (e.g., radio), and/or a receiver 230 (e.g., radio). Signals provided to and received from the transmitter 228 and the receiver 230, correspondingly, may include signaling information/data in accordance with air interface standards of applicable wireless systems. In this regard, the external computing entity 112-a may be capable of operating with one or more air interface standards, communication protocols, modulation types, and access types. More particularly, the external computing entity 112-a may operate in accordance with any of a number of wireless communication standards and protocols, such as those described above with regard to the computing entity 102.

Via these communication standards and protocols, the external computing entity 112-a may communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 112-a may also download changes, add-ons, and updates, for instance, to its firmware, software (e.g., including executable instructions, applications, program modules), operating system, and/or the like.

According to one embodiment, the external computing entity 112-a may include location determining embodiments, devices, modules, functionalities, and/or the like. Location determining features may be used, for example, to confirm a location of devices described herein for more accurately identifying or confirming an associated patient, medical care facility, residence, etc. For example, the external computing entity 112-a may include positioning embodiments, such as a location module adapted to acquire, for example, latitude, longitude, geocode, universal time (UTC), date, and/or various other information/data. The location information/data may be determined by triangulating a position of the external computing entity 112-a in connection with a variety of other systems, including cellular towers, Wi-Fi access points, and/or the like. Further, indoor location determining systems of example embodiments may use various position or location technologies including RFID tags, indoor beacons or transmitters, Wi-Fi access points, cellular towers, nearby computing devices (e.g., smartphones, laptops), and/or the like. For instance, such technologies may include the iBeacons, Gimbal proximity beacons, Bluetooth Low Energy (BLE) transmitters, NFC transmitters, and/or the like. These indoor positioning embodiments may be used in a variety of settings to determine the location of someone or something within inches or centimeters.

The external entity I/O elements 218 may include one or more external entity output devices 220 and/or one or more external entity input devices 222 that may include one or more sensory devices described herein with reference to the I/O elements 114. In some embodiments, the external entity I/O element 218 may include a user interface (e.g., a display, speaker, and/or the like) and/or a user input interface (e.g., keypad, touch screen, microphone, and/or the like) that may be coupled to the external entity processing element 210.

For example, the user interface may be a user application, browser, and/or similar words used herein interchangeably executing on and/or accessible via the external computing entity 112-a to interact with and/or cause the display, announcement, and/or the like of information/data to a user. The user input interface may include any of a number of input devices or interfaces allowing the external computing entity 112-a to receive data including, as examples, a keypad (hard or soft), a touch display, voice/speech interfaces, motion interfaces, and/or any other input device. In embodiments including a keypad, the keypad may include (or cause display of) the conventional numeric (0-9) and related keys (#, *, and/or the like), and other keys used for operating the external computing entity 112-a and may include a full set of alphabetic keys or set of keys that may be activated to provide a full set of alphanumeric keys. In addition to providing input, the user input interface may be used, for example, to activate or deactivate certain functions, such as screen savers, sleep modes, and/or the like.

### III. EXAMPLES OF CERTAIN TERMS

In some embodiments, the term “fluid collection reservoir” refers to a structure or a component that collects or otherwise contains one or more fluids. A fluid collection reservoir may be a subcomponent of a device, such as a medical device for the collection of one or more bodily fluids (e.g., a fluid collection device, a fluid measurement device). In some examples, a device may include multiple fluid collection reservoirs. For example, a chest tube cannister may include multiple, adjacent fluid collection reservoirs, such that a second fluid collection reservoir begins to fill once a first fluid collection reservoir reaches a threshold fluid level (e.g., a threshold capacity). In some examples, a fluid collection reservoir may include one or more input ports for receiving one or more fluids, for example, via a tube or any other type of fluid connecting equipment configured to deliver one or more fluids from a fluid source (e.g., an individual and/or a device) to the fluid collection reservoir. Additionally, or alternatively, a fluid collection reservoir may include an opening for receiving one or more fluids, which may be transferred to the fluid collection reservoir with or without a connecting tube. For example, a urinal (e.g., a urinal bottle) may be configured to collect and store urine from an individual by way of a catheter and/or without the use of a catheter (e.g., the individual may urinate directly into the urinal). A fluid collection reservoir may include one or more output ports or openings for draining one or more fluids from the fluid collection reservoir.

A fluid collection reservoir may be embodied in a variety of configurations and devices. In some examples, a fluid collection reservoir may be cylindrical. A cylindrical fluid collection reservoir may include a body, a base, and a lid, which may surround and otherwise contain one or more fluids. In some examples, a fluid collection reservoir may be shaped in the form of a rectangular channel. As described herein, one or more sensors, such as one or more capacitance sensors may be positioned within a fluid collection reservoir. In some examples, the one or more sensors may be physically coupled (e.g., in contact with) the fluid collection reservoir. For example, one or more capacitance sensors may be connected to an interior wall or a body of a fluid collection reservoir. In some examples, the one or more sensors may be configured to output one or more signals indicative of information associated with one or more fluids collected in the fluid collection reservoir. For example, one or more capacitance sensors may generate or otherwise output one or more signals (e.g., one or more currents, one or more voltages, one or more resistances, one or more capacitances), which may be utilized (e.g., by one or more processors) to determine information, such as one or more fluid levels (e.g., a first fluid level, a second fluid level, a third fluid level), a tilt angle of the fluid collection reservoir, whether one or more measurement accuracy criteria are satisfied, and/or the like.

In some examples, a fluid level may correspond to or otherwise be associated with a location of a sensor. For example, a first capacitance sensor may be positioned at a first location and may output a first signal indicative of a first fluid level at a first location (e.g., a location of the first capacitance sensor). Additionally, or alternatively, a second capacitance sensor may be positioned at a second location and may output a second signal indicative of a second fluid level at a second location (e.g., a location of the second capacitance sensor). In some examples, one or more processors may generate or otherwise determine one or more third fluid levels (e.g., at one or more third locations) based on a first fluid level at a first location and a second fluid level at a second location. For example, the one or more processors may average a first fluid level and a second fluid level to determine a third fluid level.

In some embodiments, the term “bodily fluid” refers to a fluid excreted by, expelled from, introduced to, in contact with, and/or withdrawn from an individual, such as a patient in a healthcare setting. As described herein, any reference to a fluid, such as a fluid in a fluid collection reservoir, may include a bodily fluid, such as blood, urine, plasma, serum, lymph, cerebrospinal fluid, synovial fluid, saliva, water, saline solution, a fluid medication, or any combination thereof. Although in some examples, the techniques described herein refer to bodily fluids, the techniques described herein may additionally, or alternatively be applied to fluids other than bodily fluids, such as cleaning fluids, synthetic lubricating agents, liquid fuels, and other fluids utilized in various contexts.

In some embodiments, the term “capacitance sensor” refers to a type of sensor configured to detect or measure one or more properties, parameters, or conditions. For example, a capacitance rod or flexible tape PSBs may output one or more capacitance values based on a fluid level along the length of the capacitance sensor. In such examples, a capacitance of the capacitance sensor may be altered when the capacitance sensor is in contact with a fluid, or a body of the capacitance sensor fills with fluid. Accordingly, one or more processors may perform one or more operations to determine a fluid level along a length of the capacitance sensor based on one or more capacitance values output by the capacitance sensor.

While illustrated and described embodiments include capacitance rods, embodiments could optionally or alternatively employ capacitance sensors of different form factors. For example, a capacitance rod may be a vertical rod to measure a fluid height along the length of the rod. However, a capacitance rod can also be embodied as a ring or series of rings disposed at various positions along a height of a container, where the capacitance ring measures the presence or absence of fluid at a location of the capacitance ring. Further, a capacitance rod could be a spiral form factor, where the rod extends vertically but also wraps around a fluid container to measure a height of fluid along the length of the capacitance rod

As described herein, a capacitance rod may include one or more capacitor plates extending along a length of the capacitance rod. In some examples, a capacitor plate may be formed in a cylindrical shape (e.g., in the shape of a rod extending along a length of the capacitance rod). In some other examples, a capacitor plate may be formed in a rectangular shape. As described herein, a capacitance value output by a capacitance rod may increase as a fluid fills the capacitance rod (e.g., fills a body of the capacitance rod) and thereby fills a gap or a space between two capacitor plates inside of the capacitance rod.

While illustrated and described embodiments include capacitance rods, embodiments could optionally or alternatively employ a flexible tape printed circuit board as a sensor type that measures an alternating current waveform signal and capacitance as a way to measure fluid levels. Such flexible tape PCBs can be employed such as in disposable applications or applications in which the fluid collection reservoir (e.g., urinal 400) are not rigid sided, such as with a catheter bag that is a flexible fluid collection reservoir. Thus, while capacitance rods are described in various embodiments herein, such flexible tape PCBs can be used to achieve substantially the same effect.

In some embodiments, the term “fluid level” refers to a degree or extent to which fluid fills a fluid collection reservoir. A fluid level may be indicative of a location along the length of a fluid level sensor, such as a capacitance rod or flexible tape PSB. The location along the length of the fluid level sensor may correspond to a location of a fluid meniscus, or a fluid-air interface. In some examples, a fluid level may indicate or correspond to a volume of fluid within a fluid collection reservoir.

In accordance with one or more examples described herein, any one or more sensors may output one or more signals indicative of or otherwise utilized to determine one or more properties, values, and/or characteristics. For example, one or more capacitance sensors may output one or more signals indicative of or otherwise corresponding to one or more capacitance values, one or more fluid level values, one or more fluid volume values, one or more tilt angles, one or more agitation values (e.g., fluid level variation within a time period), and/or any other property, value, and/or characteristic described herein. As another illustrative example, one or more pressure sensors may output one or more signals indicative of or otherwise corresponding to one or more pressure values, one or more fluid level values, one or more fluid volume values, one or more tilt angles, one or more agitation values, and/or any other property, value, and/or characteristic described herein. As yet another illustrative example, one or more weight sensors may output one or more signals indicative of or otherwise corresponding to one or more weight values, one or more fluid level values, one or more fluid volume values, one or more tilt angles, one or more agitation values, and/or any other property, value, and/or characteristic described herein.

In some examples, one or more sensor values may be used to verify and/or confirm one or more values output by one or more other sensors. For example, one or more weight sensors may be utilized in combination with one or more capacitance sensors. In such examples, a first fluid level and/or first fluid volume may be determined by one or more processors based on one or more signals output by one or more capacitance sensors. The first fluid level and/or first fluid volume may then be verified using one or more signals output by one or more weight sensors. For example, one or more processors may compare the first fluid level and/or first fluid volume determined using one or more capacitance sensors with a second fluid level and/or second fluid volume determined using one or more weight sensors. The one or more processors may then output a final fluid level and/or final fluid volume based on the first and second fluid level and/or the first and second fluid volume. In some examples, the final fluid level and/or final fluid volume may be an average of one or more fluid levels and/or fluid volumes determined using two or more types of sensors. In some other examples, the final fluid level and/or final fluid volume may be a fluid level and/or fluid volume determined using one or more first sensors that is confirmed to be within a threshold of one or more fluid levels and/or fluid volumes determined using one or more second sensors.

In some embodiments, the term “location within the fluid collection reservoir” refers to a location, point, or region of the fluid collection reservoir where a fluid level is measured. For example, a first capacitance sensor may be positioned at a first location within a fluid collection reservoir and may therefore measure a fluid level at the first location. A second capacitance sensor may be positioned at a second location with the fluid collection reservoir and may therefore measure a fluid level at the second location. In one illustrative example of a cylindrical fluid collection reservoir having a height axis extending along a first direction (e.g., a y-direction), the location may correspond to a point in a two-dimensional plane (e.g., an x-z plane).

In some embodiments, the term “health metric” refers to an indicator or a value representative of the health status or quality of an individual. For example, a quantity of urine expelled by an individual per day may be an example of a health metric. As described herein, one or more health metrics may be determined by one or more processors based on a quantity of a fluid in a fluid collection reservoir and/or based on a flow rate at which fluid enters a fluid collection reservoir. In such examples, the one or more processors may communicate or otherwise cause communication of the one or more health metrics to a user interface (e.g., a display) and/or to an electronic medical record for one or more individuals. Some other illustrative examples of health metrics include a blood drainage health metric, a pleural fluid volume health metric, a pleural pressure health metric, a blood glucose health metric, a urine color health metric, and a urine turbidity health metric, among other examples.

In some embodiments, the term “tilt angle” refers to an extent or degree to which an axis of a fluid collection reservoir deviates from a reference axis, such as a vertical axis. As one illustrative example, a tilt angle of a cylindrical fluid collection reservoir may be zero when the height axis of the cylindrical fluid collection reservoir is aligned with a vertical reference axis. In such an example, a top surface of fluid within the fluid collection reservoir may be aligned with a horizontal plane. Additionally, or alternatively, in such an example, a first fluid level measured using a first capacitance sensor at a first location within the fluid collection reservoir may be the same as a second fluid level measured using a second capacitance sensor at a second location within the fluid collection reservoir (e.g., where the first capacitance sensor and the second capacitance sensor are parallel with the height axis of the fluid collection reservoir).

In some examples, a tilt angle of a fluid collection reservoir may be utilized by one or more processors to determine one or more measurement timings for one or more fluid level measurements. For example, one or more processors may refrain from storing, calculating, or outputting one or more fluid level measurements based on one or more capacitance readings being generated during a time interval when a tilt angle of the fluid collection reservoir satisfied a threshold tilt angle (e.g., was greater than a threshold tilt angle). Additionally, or alternatively, one or more processors may perform one or more operations to filter or otherwise select one or more fluid level measurements based on a determination of whether a title angle of the fluid collection reservoir satisfies a threshold tilt angle. Accordingly, such techniques may enable the accuracy of fluid level measurements to be improved when compared to other techniques that do not account for tilt angle.

In some examples, a tilt angle may be determined by comparing two or more capacitance readings generated by two or more capacitance sensors. For example, one or more processors may determine a tilt angle of a fluid collection reservoir based on comparing two or more capacitance readings generated by two or more capacitance sensors. In such examples, a difference between a first capacitance reading (e.g., value) from a first capacitance sensor and a second capacitance reading from a second capacitance sensor may be indicative of a tilt angle greater than zero. While two capacitance sensors may be employed to indicate the possibility of a tilted container, three or more capacitance sensors may be employed to confirm the presence of container tilt, along with identifying an angle of the container tilt and direction thereof. Additionally, or alternatively, a first capacitance reading from a first capacitance sensor and a second capacitance reading from a second capacitance sensor having a same value may be indicative of a tilt angle of zero or a near zero value (e.g., within a threshold distance of zero). In such examples, the one or more processors may determine the tilt angle based on a first capacitance value output by a first capacitance sensor, a second capacitance value output by a second capacitance sensor, and a distance between the first capacitance sensor and the second capacitance sensor.

In some examples, a tilt angle may be measured by an accelerometer, which may be included in a microprocessor of a fluid measurement device. Additionally, or alternatively, one or more accelerometers may be utilized to verify a tilt angle determined based on capacitance readings from two or more capacitance sensors. For example, a tilt angle measured by an accelerometer may be utilized to calibrate one or more capacitance sensors and/or may be used in real time to verify one or more tilt angles determined based on capacitance readings. In some other examples, one or more tilt angles determined using two or more capacitance sensors may be used to verify one or more tilt angles determined using an accelerometer. Additionally, or alternatively, one or more pressure sensors may be utilized to determine one or more tilt angles. As described herein, any one or more sensors may be utilized to determine one or more tilt angles and/or to verify one or more tilt angles determined using one or more other sensors.

In some examples, a fluid measurement device may report information, such as a fluid level measurement information to one or more external devices (e.g., one or more external computing entities, one or more electronic medical records) regardless of a tilt angle of the fluid measurement device. In such examples, one or more processors of the fluid measurement device may determine a fluid level (e.g., a fluid volume) within the fluid measurement device based on the tilt angle, which enable fluid level measurements to be determined and reported without the fluid measurement device being level, which may provide advantages when compared to one or more other techniques. For example, the fluid measurement device may immediately report fluid measurements to an electronic medical record upon detecting a change in fluid level, which may be advantageous in at least one scenario where a user may discard fluid from within the fluid measurement device prior to leveling the fluid measurement device or placing the fluid measurement device on a flat surface. Such techniques may prevent the manipulation or alteration of measured fluid levels prior to recording.

In some embodiments, the term “measurement accuracy criteria” refers to one or more conditions or standards that enable a measurement to be obtained with a specific level of accuracy. For example, a specific tilt angle (e.g., a specific tilt level or value) may be utilized as a measurement accuracy criterion. In such examples, one or more processors may determine whether a tilt angle of a fluid collection reservoir satisfies a threshold (e.g., is less than the specific tilt angle). If the tilt angle of the fluid collection reservoir satisfies the threshold, the one or more processors may determine that one or more measurements (e.g., fluid level measurements) may be obtained, stored, and/or output. Additionally, or alternatively, the one or more processors may determine a measurement timing based on whether the measurement accuracy criterion is satisfied. For example, the one or more processors may determine to refrain from obtaining, storing, and/or outputting one or more fluid level measurements until the measurement accuracy criterion is satisfied.

In some embodiments, the term “electronic medical record” refers to a data log or a database that stores healthcare information for one or more individuals. In some examples, an electronic medical record or a computing entity storing one or more electronic medical records may communicate with a fluid measurement device via one or more communication components of the fluid measurement device. For example, one or more processors of a fluid measurement device may output one or more fluid level values, which may be transmitted (e.g., via one or more communication components) to one or more electronic medical records. In some examples, healthcare information for an individual, such as the one or more fluid level values, may be communicated to an electronic medical record using a specific type of message that protects or otherwise conceals at least a portion of identification information for the individual. For example, the specific type of message may include an identification number for the individual and may omit a first and last name for the individual and/or a social security number for the individual. In some examples, the specific type of message may be a health level 7 (HL7) message.

### IV. OVERVIEW

Various devices may be utilized to collect fluids for different applications. For example, fluid collection devices may be utilized for healthcare applications, industrial applications, automotive applications, agricultural applications, building system applications, and/or the like. In some examples, a fluid collection device may include one or more fluid level markings indicative of a volume of a fluid in the fluid collection device. Such fluid level markings may be utilized by one or more individuals to manually determine a fluid level within the fluid collection device. However, the manual monitoring of a fluid level within a fluid collection device may present various challenges, particularly in environments where the potential for human error poses various safety hazards, or in time-constrained environments where workforce resources are limited.

In a medical context, for example, various types of fluid collection devices may be utilized that conventionally rely on medical staff or patients to manually determine fluid level values by visual inspection of the fluid collection device. Some illustrative examples of such devices include urinals, chest tube cannisters, and wall canisters, among other examples. In such contexts, the manual determination of one or more fluid level values by an individual may present various challenges. For example, a medical professional may direct their attention away from one or more other tasks to determine one or more fluid level values, which may increase errors associated with the one or more other tasks (e.g., a medical procedure) and/or the accurate determination of the one or more fluid level values. Additionally, or alternatively, an individual may be incapable of monitoring fluid level values at a frequency that is sufficient to ensure positive health outcomes for a patient. For example, a fluid level may rapidly increase without being noticed by a medical professional, which may increase a likelihood of adverse health effects for a patient.

As another illustrative example, some conventional fluid collection systems may rely on a medical professional to read fluid level information from the fluid collection system and manually copy the information to a medical record for a patient. However, such techniques may be time consuming and may be subject to human error. Additionally, such techniques may be limited in that a medical professional may not be capable of recording fluid level values at a frequency necessary to identify various health conditions or health risks. Accordingly, such techniques may result in negative health outcomes for patients.

The systems and techniques described herein enable the automatic measurement and reporting of fluid level values, which may improve patient health outcomes. In accordance with one or more examples described herein, a fluid measurement device is provided. The fluid measurement device may include a fluid collection reservoir and one or more capacitance sensors positioned in the fluid collection reservoir. The one or more capacitance sensors may be configured to output one or more signals to one or more processors, which may be located in a base of the fluid measurement device. The one or more processors may receive the one or more signals and may determine information based on the one or more signals. For example, the one or more processors may determine a tilt angle of the fluid measurement device and/or one or more fluid levels of fluid in the fluid collection reservoir. In some examples, the one or more fluid levels may be determined by the one or more processors based on the tilt angle. In some examples, the one or more processors may determine whether one or more measurement accuracy criteria are satisfied (e.g., based on the one or more signals received from the one or more capacitance sensors). In such examples, the one or more processors may determine one or more measurement timings (e.g., for determining one or more fluid levels) based on whether the one or more measurement accuracy criteria are satisfied. Accordingly, the one or more processors may avoid determining, generating, or outputting one or more fluid level values if the fluid measurement device has been agitated, if the fluid measurement device is tilted, and/or if an error has occurred (e.g., if a capacitance sensor fails to output a signal), which may improve measurement accuracy.

By enabling the automatic measurement and reporting of fluid level values, the systems and methods described herein may result in improved patient health outcomes. Additionally, the systems and techniques described herein enable accurate measurement data to be obtained in a variety of conditions and environments. For example, various combinations of sensors and processing techniques may be utilized to provide accurate fluid level measurements even if a fluid collection device is tilted or agitated. Additionally, or alternatively, the utilization of two or more capacitance sensors to determine tilt angle may enable the determination of tilt angle without the use of one or more additional sensors (e.g., an accelerometer) for measuring tilt angle, which may reduce complexity of a fluid measurement device, thereby reducing cost and improving reliability.

In some examples, the fluid measurement device may communicate one or more fluid level values to a medical record or to a computing entity that stores a medical record. Such communications may enable health data for one or more individuals to be recorded more efficiently when compared to conventional techniques that involve manual recordation of data. In some examples, the fluid measurement device may communicate the one or more fluid level values to the medical record via a specific type of message that protects the identity of a patient (e.g., names and social security numbers of patients are not included in the message). For example, the fluid measurement device may communicate the one or more fluid level values to the medical record using a health level 7 (HL7) message. In such examples, the fluid measurement device may receive a patient identifier (e.g., other than a name or social security number for the patient) and may link the patient identifier to the one or more fluid level values. In some examples, a healthcare provider may then retrieve the data and prescribe a treatment and/or medication to the patient based on the data. The patient may then perform one or more actions (e.g., adhere to a treatment plan), which may result in an improved health outcome for the patient.

Examples of technologically advantageous embodiments of the present disclosure include techniques for efficiently and securely monitoring and communicating healthcare information for one or more individuals, among other examples. Other technical improvements and advantages may be realized by one of ordinary skill in the art.

### V. EXAMPLE SYSTEM FRAMEWORKS

As indicated, various embodiments of the present disclosure make important technical contributions to the field of healthcare. In particular, systems and methods are disclosed herein that enable collected fluid levels to be accurately measured and communicated to an electronic medical record. When compared to traditional techniques, monitoring techniques described herein provide increased monitory accuracy, thereby improving patient health outcomes.

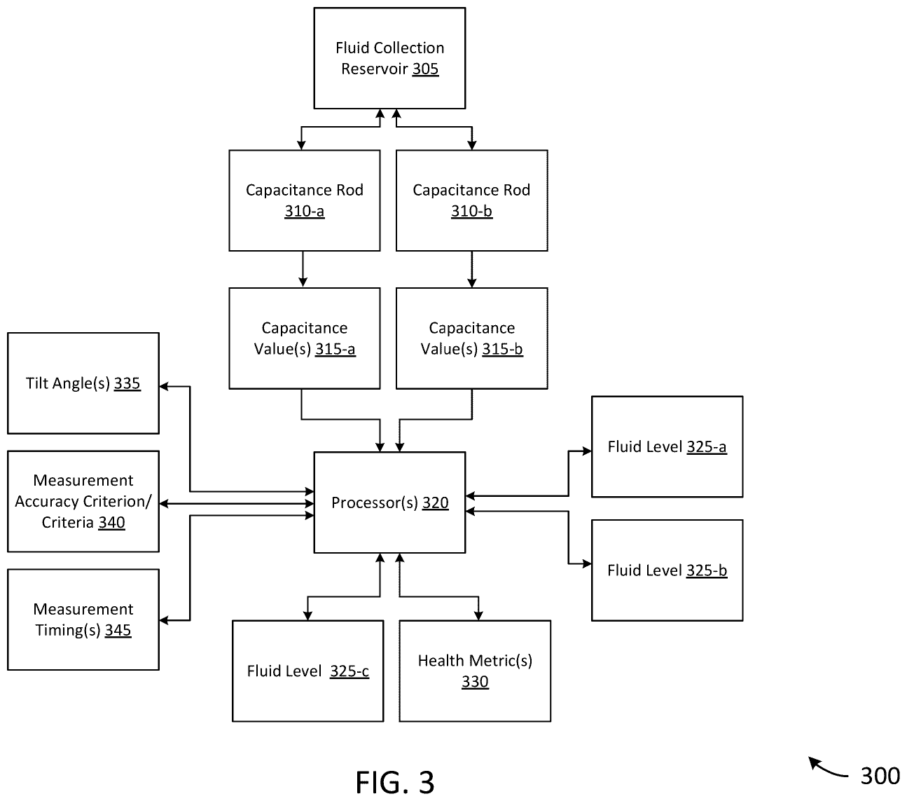

FIG. 3 illustrates a data flow diagram 300 in accordance with some embodiments discussed herein. The data flow diagram 300 may illustrate examples of one or more operations, which may be performed by any one or more devices or components as described herein, such as a fluid measurement device, one or more processors 320 of a fluid measurement device, one or more capacitance sensors 310, and/or the like. The data flow diagram 300 may provide one or more examples of data types, which may be generated, communicated, input, output, and/or the like by one or more components of a fluid measurement device.

In one embodiment, a capacitance sensor 310-a disposed in a fluid collection reservoir 305 may output a capacitance value 315-a indicative of a fluid level 325-a of one or more bodily fluids at a first location within the fluid collection reservoir. In some examples, the one or more bodily fluids may be associated with an individual. In one embodiment, a capacitance sensor 310-b disposed in the fluid collection reservoir 305 may output a capacitance value 315-b indicative of a fluid level 325-b of the one or more bodily fluids at a second location within the fluid collection reservoir 305.

In some examples, a fluid collection reservoir 305 is a structure or a component that collects or otherwise contains one or more fluids. A fluid collection reservoir 305 may be a subcomponent of a device, such as a medical device for the collection of one or more bodily fluids (e.g., a fluid collection device, a fluid measurement device). In some examples, a device may include multiple fluid collection reservoirs 305. For example, a chest tube cannister may include multiple, adjacent fluid collection reservoirs 305, such that a second fluid collection reservoir 305 begins to fill once a first fluid collection reservoir 305 reaches a threshold fluid level (e.g., a threshold capacity).

In some examples, a fluid collection reservoir 305 may include one or more input ports for receiving one or more fluids, for example, via a tube or any other type of fluid connecting equipment configured to deliver one or more fluids from a fluid source (e.g., an individual and/or a device) to the fluid collection reservoir 305. Additionally, or alternatively, a fluid collection reservoir 305 may include an opening for receiving one or more fluids, which may be transferred to the fluid collection reservoir 305 with or without a connecting tube. For example, a urinal (e.g., a urinal bottle) may be configured to collect and store urine from an individual by way of a catheter and/or without the use of a catheter (e.g., the individual may urinate directly into the urinal). A fluid collection reservoir 305 may include one or more output ports or openings for draining one or more fluids from the fluid collection reservoir 305.

A fluid collection reservoir 305 may be embodied in a variety of configurations and devices. In some examples, a fluid collection reservoir 305 may be cylindrical. A cylindrical fluid collection reservoir 305 may include a body, a base, and a lid, which may surround and otherwise contain one or more fluids. In some examples, a fluid collection reservoir 305 may be shaped in the form of a rectangular channel. As described herein, one or more sensors, such as one or more capacitance sensors 310 may be positioned within a fluid collection reservoir 305. In some examples, the one or more sensors may be physically coupled (e.g., in contact with) the fluid collection reservoir 305. For example, one or more capacitance sensors 310 may be connected to an interior wall or a body of a fluid collection reservoir 305.

In some examples, such as in the example of a chest tube cannister, a capacitance sensor 310 or plate or flexible tape PSB may form one or more walls of a fluid collection reservoir 305. In some examples, the one or more sensors may be configured to output one or more signals indicative of information associated with one or more fluids collected in the fluid collection reservoir 305. For example, one or more capacitance sensors 310 may generate or otherwise output one or more signals (e.g., one or more currents, one or more voltages, one or more resistances, one or more capacitances), which may be utilized (e.g., by one or more processors 320) to determine information, such as one or more fluid levels 325 (e.g., a fluid level 325-a, a fluid level 325-b, a fluid level 325-c), a tilt angle 335 of the fluid collection reservoir 305, whether one or more measurement accuracy criteria 340 are satisfied, and/or the like.

In some examples, a fluid level 325 may correspond to or otherwise be associated with a location of a sensor. For example, a capacitance sensor 310-a may be positioned at a first location and may output a first signal indicative of a fluid level 325-a at a first location (e.g., a location of the capacitance sensor 310-a). Additionally, or alternatively, a capacitance sensor 310-b may be positioned at a second location and may output a second signal indicative of a fluid level 325-b at a second location (e.g., a location of the capacitance sensor 310-b). In some examples, one or more processors 320 may generate or otherwise determine one or more third fluid levels 325-c at one or more third locations based on a fluid level 325-a at a first location and a fluid level 325-b at a second location. For example, the one or more processors 320 may average a fluid level 325-a and a fluid level 325-b to determine a fluid level 325-c.

In some examples, a bodily fluid is a fluid excreted by, expelled from, introduced to, in contact with, and/or withdrawn from an individual, such as a patient in a healthcare setting. As described herein, any reference to a fluid, such as a fluid in a fluid collection reservoir 305, may include a bodily fluid, such as blood, urine, plasma, serum, lymph, cerebrospinal fluid, synovial fluid, saliva, water, saline solution, a fluid medication, or any combination thereof. Although in some examples, the techniques described herein refer to bodily fluids, the techniques described herein may additionally, or alternatively be applied to fluids other than bodily fluids, such as cleaning fluids, synthetic lubricating agents, and other fluids utilized in various other contexts.

In some examples, a capacitance sensor 310 is a type of sensor configured to detect or measure one or more properties, parameters, or conditions. For example, a capacitance sensor 310 may output one or more capacitance values 315 based on a fluid level 325 along the length of the capacitance sensor 310. In such examples, a capacitance of the capacitance sensor 310 may be altered when the capacitance sensor 310 is in contact with fluid or a body of the capacitance sensor 310 fills with fluid. Accordingly, one or more processors 320 may perform one or more operations to determine a fluid level 325 along a length of the capacitance sensor 310 based on one or more capacitance values 315 output by the capacitance sensor 310.

As described herein, a capacitance sensor 310 may include one or more capacitor plates extending along a length of the capacitance sensor 310. In some examples, a capacitor plate may be formed in a cylindrical shape (e.g., in the shape of a rod extending along a length of the capacitance rod 310). In some other examples, a capacitor plate may be formed in a rectangular shape. As described herein, a capacitance value 315 output by a capacitance sensor 310 may increase as a fluid fills the capacitance sensor 310 (e.g., fills a body of the capacitance sensor 310) and thereby fills a gap or a space between two capacitor plates inside of the capacitance sensor 310.

In some examples, a fluid level 325 is a degree or extent to which fluid fills a fluid collection reservoir 305. A fluid level 325 may be indicative of a location along the length of a fluid level sensor, such as a capacitance sensor 310. The location along the length of the fluid level sensor may correspond to a location of a fluid meniscus, or a fluid-air interface. In some examples, a fluid level 325 may indicate or correspond to a volume of fluid within a fluid collection reservoir 305.

In some examples, a location within a fluid collection reservoir 305 is a location, point, or region of the fluid collection reservoir 305 where a fluid level 325 is measured. For example, a capacitance sensor 310-a may be positioned at a first location within a fluid collection reservoir 305 and may therefore measure a fluid level 325-a at the first location. A capacitance sensor 310-b may be positioned at a second location with the fluid collection reservoir 305 and may therefore measure a fluid level 325-b at the second location. In one illustrative example of a cylindrical fluid collection reservoir 305 having a height axis extending along a first direction (e.g., a y-direction), the location may correspond to a point in a two-dimensional plane (e.g., an x-z plane).

In one embodiment, one or more processors 320 may receive the capacitance value 315-a and the capacitance value 315-b. In one embodiment, the one or more processors 320 may determine a fluid level 325-c of the one or more bodily fluids based at least in part on the fluid level 325-a and the fluid level 325-b. In some examples, the fluid level 325-c may be indicative of one or more health metrics 330 for an individual. In some examples, a health metric 330 is an indicator or a value representative of the health status or quality of an individual. For example, a quantity of urine expelled by an individual per day may be an example of a health metric 330.

As described herein, one or more health metrics 330 may be determined by one or more processors 320 based on a quantity of a fluid in a fluid collection reservoir 305 and/or based on a flow rate at which fluid enters a fluid collection reservoir 305. In such examples, the one or more processors 320 may communicate or otherwise cause communication of the one or more health metrics 330 to a user interface (e.g., a display) and/or to an electronic medical record for one or more individuals. Some other illustrative examples of health metrics 330 include a blood drainage health metric, a pleural fluid volume health metric, a pleural pressure health metric, a blood glucose health metric, a urine color health metric, and a urine turbidity health metric, among other examples.

In one embodiment, the one or more processors 320 may determine a tilt angle 335 of the fluid collection reservoir 305 based at least in part on a comparison of the capacitance value 315-a and the capacitance value 315-b. In some examples, the fluid level 325-c may be based at least in part on the tilt angle 335. In some examples, a tilt angle 335 is an extent or degree to which an axis of a fluid collection reservoir 305 deviates from a reference axis, such as a vertical axis. As one illustrative example, a tilt angle 335 of a cylindrical fluid collection reservoir 305 may be zero when the height axis of the cylindrical fluid collection reservoir 305 is aligned with a vertical reference axis. In such an example, a top surface of fluid within the fluid collection reservoir 305 may be aligned with a horizontal plane. Additionally, or alternatively, in such an example, a fluid level 325-a measured using a capacitance sensor 310-a at a first location within the fluid collection reservoir 305 may be the same as a fluid level 325-b measured using a capacitance sensor 310-b at a second location within the fluid collection reservoir 305 (e.g., where the capacitance sensor 310-a and the capacitance sensor 310-b are parallel with the height axis of the fluid collection reservoir 305).

In some examples, a tilt angle 335 of a fluid collection reservoir 305 may be utilized by one or more processors 320 to determine one or more measurement timings 345 for one or more fluid level 325 measurements. For example, one or more processors 320 may refrain from storing, calculating, or outputting one or more fluid level 325 measurements based on one or more capacitance readings being generated during a time interval when a tilt angle 335 of the fluid collection reservoir 305 satisfied a threshold tilt angle 335 (e.g., was greater than a threshold tilt angle 335). Additionally, or alternatively, one or more processors 320 may perform one or more operations to filter or otherwise select one or more fluid level 325 measurements based on a determination of whether a title angle of the fluid collection reservoir 305 satisfies a threshold tilt angle 335. Accordingly, such techniques may enable the accuracy of fluid level 325 measurements to be improved when compared to other techniques that do not account for tilt angle 335.

In some examples, a tilt angle 335 may be determined by comparing two or more capacitance readings generated by two or more capacitance sensors 310. For example, one or more processors 320 may determine a tilt angle 335 of a fluid collection reservoir 305 based on comparing two or more capacitance readings generated by two or more capacitance sensors 310. In such examples, a difference between a first capacitance reading (e.g., value) from a capacitance sensor 310-a and a second capacitance reading from a capacitance sensor 310-b may be indicative of a tilt angle 335 greater than zero. Additionally, or alternatively, a first capacitance reading from a capacitance sensor 310-a and a second capacitance reading from a capacitance sensor 310-b having a same value may be indicative of a tilt angle 335 of zero or a near zero value (e.g., within a threshold distance of zero). In such examples, the one or more processors 320 may determine the tilt angle 335 based on a capacitance value 315-a output by a capacitance sensor 310-a, a capacitance value 315-b output by a capacitance sensor 310-b, and a distance between the capacitance sensor 310-a and the capacitance sensor 310-b.

In one embodiment, the one or more processors 320 may determine whether one or more measurement accuracy criteria 340 are satisfied based at least in part on the capacitance value 315-a and the capacitance value 315-b. Additionally, or alternatively, the one or more processors 320 may determine a measurement timing 345 for determining the fluid level 325-c based at least in part on whether the one or more measurement accuracy criteria 340 are satisfied.

In some examples, a measurement accuracy criterion 340 is a condition or standard that enables a measurement to be obtained with a specific level of accuracy. For example, a specific tilt angle 335 (e.g., a specific tilt level or value) may be utilized as a measurement accuracy criterion 340. In such examples, one or more processors 320 may determine whether a tilt angle 335 of a fluid collection reservoir 305 satisfies a threshold (e.g., is less than the specific tilt angle 335). If the tilt angle 335 of the fluid collection reservoir 305 satisfies the threshold, the one or more processors 320 may determine that one or more measurements (e.g., fluid level 325 measurements) may be obtained, stored, and/or output. Additionally, or alternatively, the one or more processors 320 may determine a measurement timing 345 based on whether the measurement accuracy criterion 340 is satisfied. For example, the one or more processors 320 may determine to refrain from obtaining, storing, and/or outputting one or more fluid level 325 measurements until the measurement accuracy criterion 340 is satisfied.

In one embodiment, the one or more processors 320 may generate a health level 7 (HL7) message comprising at least an indication of the fluid level 325-c. Additionally, or alternatively, the one or more processors 320 may cause transmission of the HL7 message to an electronic medical record for the individual. Additionally, or alternatively, the one or more processors 320 may receive an identification number for the individual and the electronic medical record for the individual may be identified based at least in part on the identification number for the individual.

In some examples, an electronic medical record is a data log or a database that stores healthcare information for one or more individuals. In some examples, an electronic medical record or a computing entity storing one or more electronic medical records may communicate with a fluid measurement device via one or more communication components of the fluid measurement device. For example, one or more processors 320 of a fluid measurement device may output one or more fluid level 325 values, which may be transmitted (e.g., via one or more communication components) to one or more electronic medical records. In some examples, healthcare information for an individual, such as the one or more fluid level 325 values, may be communicated to an electronic medical record using a specific type of message that protects or otherwise conceals at least a portion of identification information for the individual. For example, the specific type of message may include an identification number for the individual and may omit a first and last name for the individual and/or a social security number for the individual. In some examples, the specific type of message may be a health level 7 (HL7) message. The communication of information to a medical record may include formatting of information in a specific format consistent with an electronic medical record system. The formatting of information can include manipulation of data fields to comply with medical privacy rules, digital privacy rules, and medical record requirements. In this manner, the specifically formatted messages can be encoded according to a security protocol, such as a secure key exchange, with the electronic medical record system.

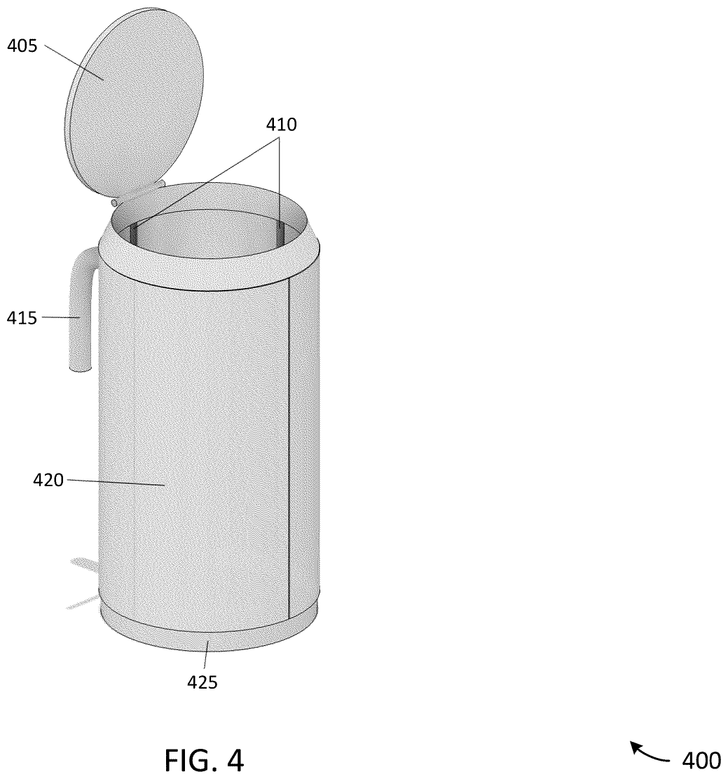

FIG. 4 illustrates an operational example of a urinal 400 in accordance with some embodiments discussed herein. As described herein, FIG. 4 may illustrate a side view of the urinal 400. The urinal 400 may be an example of a fluid measurement device configured to measure one or more bodily fluids, such as urine. The urinal 400 may include a computing entity or one or more components of computing entity, which may be utilized to perform any of the techniques described herein. For example, the urinal 400 may include one or more processors (e.g., a microcontroller), one or more communication interfaces (e.g., a receiver, a transmitter, and/or a transceiver), and one or more I/O elements (e.g., one or more user interfaces).

The urinal 400 may include one or more capacitance sensors 410, which may be utilized to measure one or more fluid levels within the urinal 400 and/or one or more tilt angles of the urinal 400. In some examples, the one or more processors of the urinal 400 may determine whether one or more measurement accuracy criteria have been satisfied based on one or more values output by the one or more capacitance sensors 410. For example, a plurality of fluid level measurements output by the one or more capacitance sensors 410 within a time interval may indicate that the urinal 400 has not been agitated within the time interval. Such information may be utilized to determine that an agitation criterion (e.g., a measurement accuracy criterion) is satisfied.

As described herein, the urinal 400 may include a base 425, which may include the computing entity or one or more components of the computing entity. Additionally, or alternatively, the urinal 400 may include a body 420 that is physically coupled with the base 425, a handle 415 that is physically coupled with the body 420, and a lid 405 that is physically coupled with the body 420. As described herein, the urinal 400, or one or more components of the urinal 400 may be an example of a fluid collection reservoir. For example, the base 425, the body 420, and the lid 405, in combination, may be an example of a fluid collection reservoir. In some examples, the body 420 may be configured to be detachable from the base 425 and/or the one or more capacitance sensors 410 (e.g., the one or more capacitance sensors 410 may be physically coupled with the base 425).

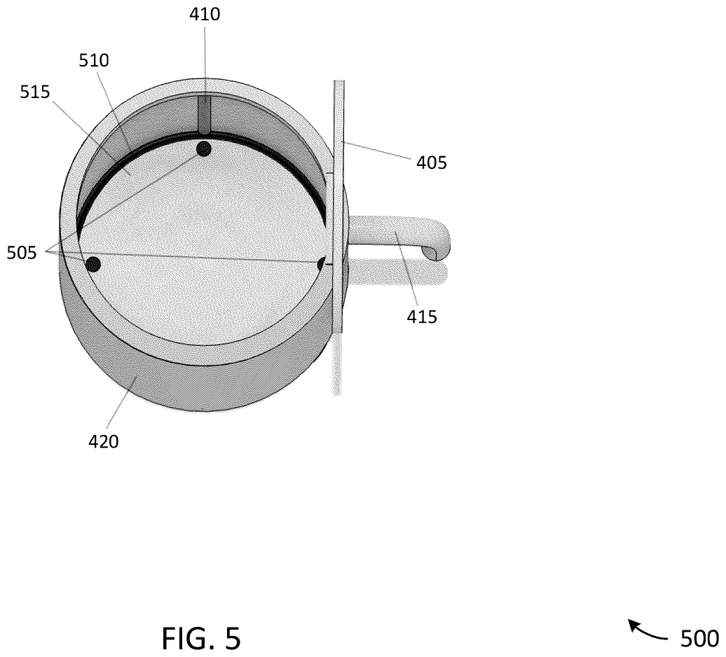

FIG. 5 illustrates an operational example of a urinal 500 in accordance with some embodiments discussed herein. The urinal 500 may be an example of the urinal 400, described with reference to FIG. 4. As described herein, FIG. 5 may illustrate a top view of the urinal 500. The urinal 500 may be an example of a fluid measurement device configured to measure one or more bodily fluids, such as urine. The urinal 500 may include a computing entity or one or more components of computing entity, which may be utilized to perform any of the techniques described herein. For example, the urinal 500 may include one or more processors (e.g., a microcontroller), one or more communication interfaces (e.g., a receiver, a transmitter, and/or a transceiver), and one or more I/O elements (e.g., one or more user interfaces).

The urinal 500 may include one or more capacitance sensors 410, which may be utilized to measure one or more fluid levels within the urinal 500 and/or one or more tilt angles of the urinal 500. Additionally, or alternatively, the urinal 500 may include one or more pressure sensors, such as one or more point pressure sensors 505 and/or one or more circumferential pressure sensors 510. In some examples, a point pressure sensor 505 and/or a circumferential pressure sensor 510 may be configured to output a signal (e.g., to the one or more processors) indicative of a fluid level or a fluid pressure at a location of the pressure sensor (e.g., above the point pressure sensor 505). In some examples, the circumferential pressure sensor 510 may be physically coupled with one or more weight scale components 515 (e.g., a round plate), which may enable the circumferential pressure sensor 510 to weigh or otherwise output a signal indicative of a weight of a fluid in the urinal 500.

In some examples, the one or more processors of the urinal 500 may determine whether one or more measurement accuracy criteria have been satisfied based on one or more values output by the one or more capacitance sensors 410 and/or the one or more pressure sensors. For example, a plurality of fluid level measurements output by the one or more capacitance sensors 410 within a time interval may indicate that the urinal 500 has not been agitated within the time interval. Such information may be utilized to determine that an agitation criterion (e.g., a measurement accuracy criterion) is satisfied.

As described herein, the urinal 500 may include a base (not shown), which may include the computing entity or one or more components of the computing entity. Additionally, or alternatively, the urinal 500 may include a body 420 that is physically coupled with the base, a handle 415 that is physically coupled with the body 420, and a lid 405 that is physically coupled with the body 420. As described herein, the urinal 500, or one or more components of the urinal 500 may be an example of a fluid collection reservoir. For example, the base, the body 420, and the lid 405, in combination, may be an example of a fluid collection reservoir. In some examples, the body 420 may be configured to be detachable from the base, the one or more pressure sensors, and/or the one or more capacitance sensors 410.

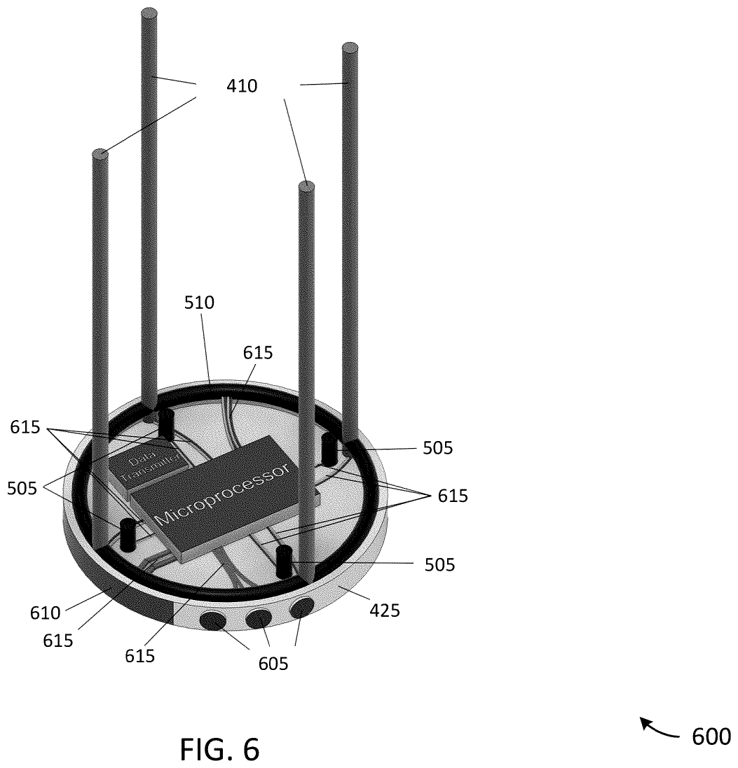

FIG. 6 illustrates an operational example of an electronics assembly 600 in accordance with some embodiments discussed herein. The electronics assembly 600 may include a base 425 of a fluid measurement device, which may house or be physically coupled with one or more other components of the electronics assembly 600. The electronics assembly 600 may include one or more processors (e.g., one or more microprocessors) and one or more communication interfaces (e.g., a data transmitter, a data receiver, a data transceiver) in communication with the one or more processors. The one or more processors may communicate information, such as one or more fluid levels to the one or more communication interfaces wirelessly or via a wired connection. The one or more communication interfaces may then communicate the information to an electronic medical record and/or to one or more user devices (e.g., a phone, a smartwatch, a tablet, a computing entity). In some examples, the one or more processors may provide the information to a display 610, which may be viewable by a user, such as a medical professional and/or a patient. In such examples, the information may be communicated from the one or more processors to the display 610 via one or more wires 615, or any other type of electrical connection.

The electronics assembly 600 may include one or more capacitance sensors 410, one or more point pressure sensors 505, and/or one or more circumferential pressure sensors 510, which may be electrically coupled with the one or more processors via one or more wires 615. Additionally, or alternatively, the electronics assembly 600 may include one or more buttons 605, which may enable a user to control and/or provide information to the one or more processors. For example, pressing a button 605 may cause the urinal (e.g., the one or more processors) to power on, power off, display a battery life reading, and/or the like. Additionally, or alternatively, a user may control one or more functions of the urinal via the one or more buttons 605, such as selecting information displayed via the display 610, causing information to be stored or communicated, causing a measurement to be initiated, setting a clock, setting a timer for initiating one or more measurements, setting a data reporting interval for reporting data to a medical record, responding to one or more prompts, and/or the like.

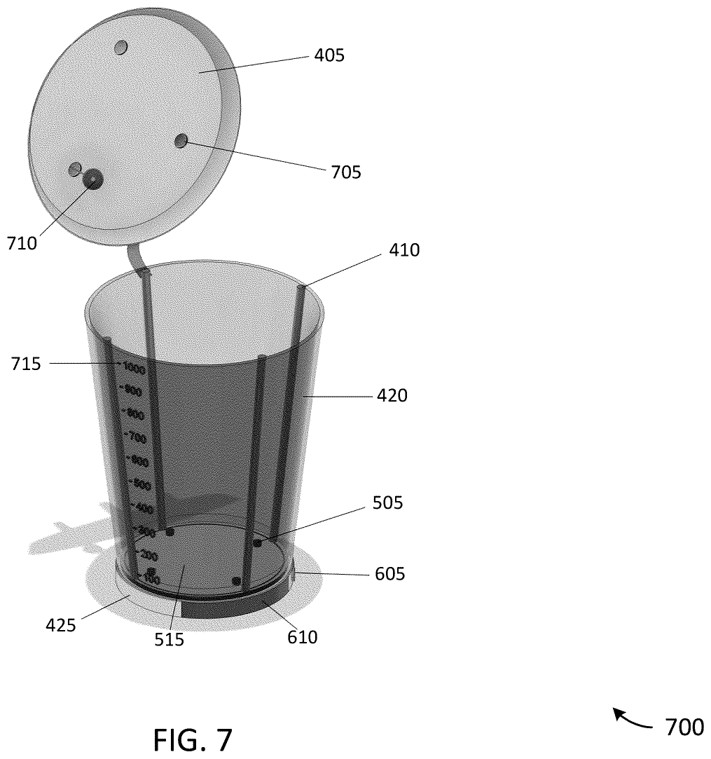

FIG. 7 illustrates an operational example of a wall cannister 700 in accordance with some embodiments discussed herein. As described herein, FIG. 7 may illustrate a side view of the wall cannister 700. The wall cannister 700 may be an example of a fluid measurement device configured to measure one or more bodily fluids, such as one or more fluids expelled by a patient during a medical procedure. The wall cannister 700 may include a computing entity or one or more components of computing entity, which may be utilized to perform any of the techniques described herein. For example, the wall cannister 700 may include one or more processors (e.g., a microcontroller), one or more communication interfaces (e.g., a receiver, a transmitter, and/or a transceiver), and one or more I/O elements (e.g., one or more user interfaces).

The wall cannister 700 may include one or more capacitance sensors 410, which may be utilized to measure one or more fluid levels within the wall cannister 700 and/or one or more tilt angles of the wall cannister 700. Additionally, or alternatively, wall cannister 700 may include one or more pressure sensors, such as one or more point pressure sensors 505 and/or one or more circumferential pressure sensors (not shown). In some examples, a point pressure sensor 505 and/or a circumferential pressure sensor may be configured to output a signal (e.g., to the one or more processors) indicative of a fluid level or a fluid pressure at a location associated with the point pressure sensor 505 (e.g., a location above the point pressure sensor 505). In some examples, the circumferential pressure sensor may be physically coupled with one or more weight scale components 515 (e.g., a round plate), which may enable the circumferential pressure sensor to weigh or otherwise output a signal indicative of a weight of a fluid in the wall cannister 700.

In some examples, the one or more processors of the wall cannister 700 may determine whether one or more measurement accuracy criteria have been satisfied based on one or more values output by the one or more capacitance sensors 410 and/or the one or more pressure sensors. For example, a plurality of fluid level measurements output by the one or more capacitance sensors 410 within a time interval may indicate that the wall cannister 700 has not been agitated within the time interval. Such information may be utilized to determine that an agitation criterion (e.g., a measurement accuracy criterion) is satisfied.

As described herein, the wall cannister 700 may include a base 425, which may include the computing entity or one or more components of the computing entity. Additionally, or alternatively, the wall cannister 700 may include a body 420 that is physically coupled with the base 425, and a lid 405 that is physically coupled with the body 420. The lid 405 may include one or more openings that allow fluid to enter or exit the wall cannister 700. For example, the lid 405 may include one or more entry holes 705. In some examples, the lid may include one or more overflow shut off ball valves 710, which may be an example of a float valve. In some examples, the body 420 may include fluid level measurement values 715, which may be inscribed or printed onto the body 420.

Additionally, or alternatively, the base 425 may include one or more buttons 605 and a display 610. As described herein, the wall cannister 700, or one or more components of the wall cannister 700 may be an example of a fluid collection reservoir. For example, the base 425, the body 420, and the lid 405, in combination, may be an example of a fluid collection reservoir. In some examples, the body 420 may be configured to be detachable from the base, the one or more pressure sensors, and/or the one or more capacitance sensors 410.

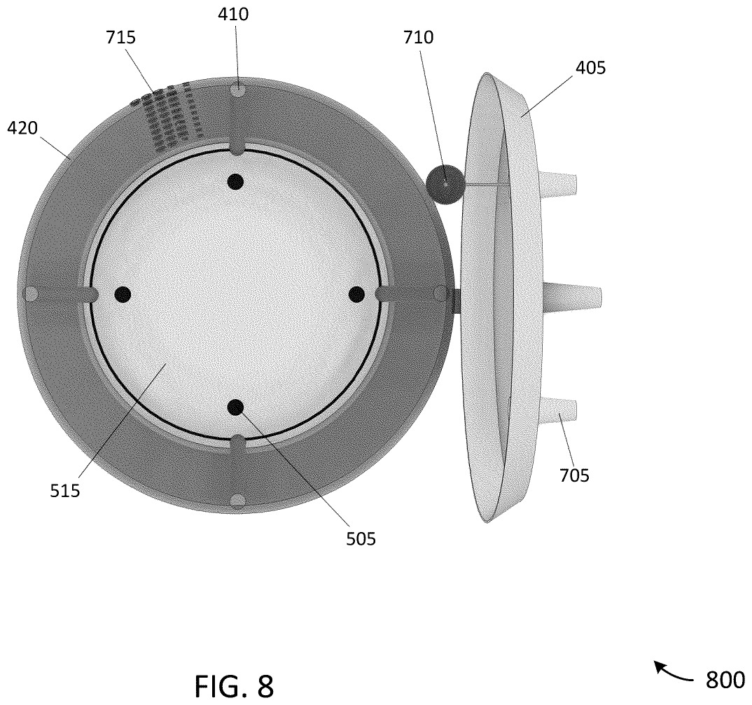

FIG. 8 illustrates an operational example of a wall cannister 800 in accordance with some embodiments discussed herein. The wall cannister 800 may be an example of the wall cannister 700, described with reference to FIG. 7. As described herein, FIG. 8 may illustrate a top view of the wall cannister 800. The wall cannister 800 may be an example of a fluid measurement device configured to measure one or more bodily fluids, such as one or more fluids expelled by a patient during a medical procedure. The wall cannister 700 may include one or more capacitance sensors 410, one or more point pressure sensors 505, one or more circumferential pressure sensors, one or more weight scale components 515, a base, a body 420, a lid 405, one or more entry holes 705, one or more overflow shut off ball valves 710, one or more fluid level measurement values 715, one or more buttons 605, and/or a display, which may be examples of corresponding elements described with reference to FIG. 7.

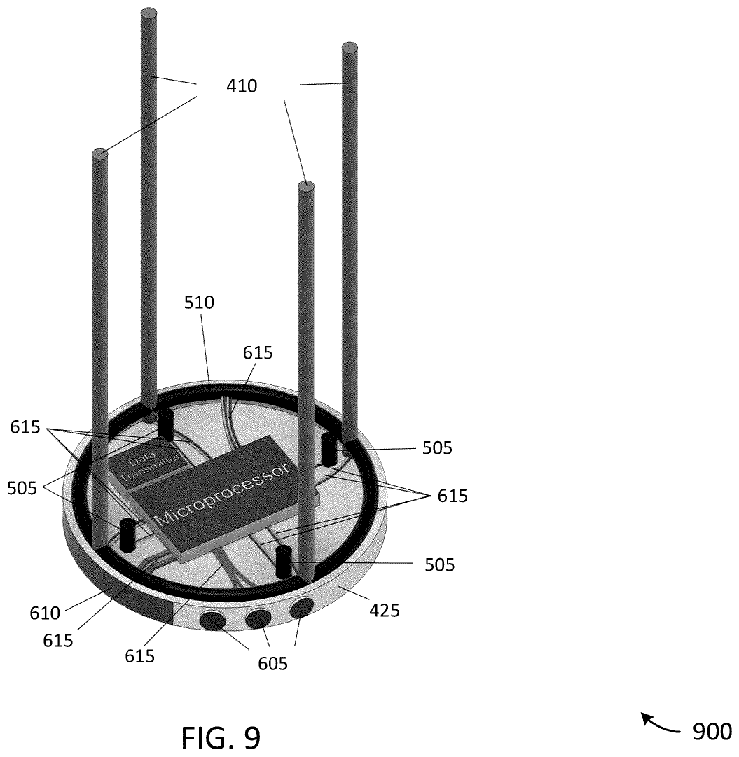

FIG. 9 illustrates an operational example of an electronics assembly 900 in accordance with some embodiments discussed herein. The electronics assembly 900 may include a base 425 of a fluid measurement device, which may house or be physically coupled with one or more other components of the electronics assembly 900. The electronics assembly 900 may include one or more processors (e.g., one or more microprocessors) and one or more communication interfaces (e.g., a data transmitter, a data receiver, a data transceiver) in communication with the one or more processors. The one or more processors may communicate information, such as one or more fluid levels to the one or more communication interfaces wirelessly or via a wired connection. The one or more communication interfaces may then communicate the information to an electronic medical record and/or to one or more user devices (e.g., a phone, a smartwatch, a tablet, a computing entity). In some examples, the one or more processors may provide the information to a display 610, which may be viewable by a user, such as a medical professional and/or a patient. In such examples, the information may be communicated from the one or more processors to the display 610 via one or more wires 615, or any other type of electrical connection.

The electronics assembly 900 may include one or more capacitance sensors 410, one or more point pressure sensors 505, and/or one or more circumferential pressure sensors 510, which may be electrically coupled with the one or more processors via one or more wires 615. Additionally, or alternatively, the electronics assembly 900 may include one or more buttons 605, which may enable a user to control and/or provide information to the one or more processors. For example, pressing a button 605 may cause the wall cannister (e.g., the one or more processors) to power on, power off, display a battery life reading, and/or the like. Additionally, or alternatively, a user may control one or more functions of the wall cannister via the one or more buttons 605, such as selecting information displayed via the display 610, causing information to be stored or communicated, causing a measurement to be initiated, setting a clock, setting a timer for initiating one or more measurements, setting a data reporting interval for reporting data to a medical record, responding to one or more prompts, and/or the like.

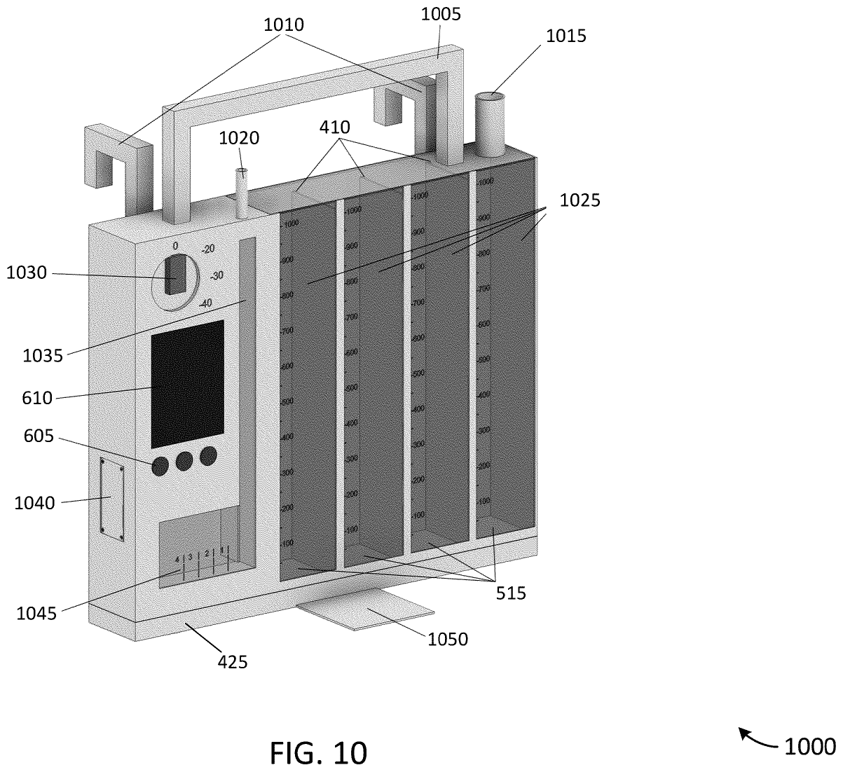

FIG. 10 illustrates an operational example of a chest tube cannister 1000 in accordance with some embodiments discussed herein. As described herein, FIG. 10 may illustrate a side view of the chest tube cannister 1000. The chest tube cannister 1000 may be an example of a fluid measurement device configured to measure one or more bodily fluids, such as one or more fluids expelled from a chest cavity of a patient during a medical procedure. The chest tube cannister 1000 may include a computing entity or one or more components of computing entity, which may be utilized to perform any of the techniques described herein. For example, the chest tube cannister 1000 may include one or more processors (e.g., a microcontroller), one or more communication interfaces (e.g., a receiver, a transmitter, and/or a transceiver), and one or more I/O elements (e.g., one or more user interfaces).

The chest tube cannister 1000 may include one or more capacitance sensors 410, which may be utilized to measure one or more fluid levels within the chest tube cannister 1000 and/or one or more tilt angles of the chest tube cannister 1000. The chest tube cannister 1000 may include one or more fluid collection chambers 1025 (e.g., fluid collection reservoirs). In some examples, the fluid collection chambers 1025 may be configured to fill sequentially. For example, once a first fluid collection chamber 1025 of the chest tube cannister 1000 reaches a threshold fill level, a second fluid collection chamber 1025 of the chest tube cannister 1000 may begin to fill. Once the second fluid collection chamber 1025 of the chest tube cannister 1000 reaches a threshold fill level, a third fluid collection chamber 1025 of the chest tube cannister 1000 may begin to fill, and so forth. As described herein, each fluid collection chamber 1025 may include one or more capacitance sensors 410.

In some examples, the chest tube cannister 1000 may include one or more pressure sensors, such as one or more point pressure sensors (not shown). In such examples, a point pressure sensor may be positioned below and/or physically coupled with a weight scale component 515 (e.g., a square or rectangular plate). A fluid in a fluid collection chamber 1025 may exert a downward force on the weight scale component 515, which may cause a point pressure sensor to output a signal indicative of weight or volume of the fluid. For example, a signal and/or a pressure value may be utilized by one or more processors to determine or otherwise calculate a volume of the fluid (e.g., based on one or more properties of the fluid).

In some examples, the one or more processors of the chest tube cannister 1000 may determine whether one or more measurement accuracy criteria have been satisfied based on one or more values output by the one or more capacitance sensors 410 and/or the one or more pressure sensors. For example, a plurality of fluid level measurements output by the one or more capacitance sensors 410 within a time interval may indicate that the chest tube cannister 1000 has not been agitated within the time interval. Such information may be utilized to determine that an agitation criterion (e.g., a measurement accuracy criterion) is satisfied.

As described herein, the chest tube cannister 1000 may include a base 425, which may include the computing entity or one or more components of the computing entity. The chest tube cannister 1000 may include a stabilizing component 1050, which may be physically coupled with the base 425 and may enable the chest tube cannister 1000 to be positioned on a flat surface, such as a table or a floor. In some examples, the chest tube cannister 1000 may be carried or lifted by a user by way of a handle 1005. In some examples, the chest tube cannister 1000 may be hung from one or more other objects via the one or more hangers 1010.

The chest tube cannister 1000 may include one or more openings, such as a fluid entry opening 1015 (e.g., a chest tube connector origin). In some examples, a tube or connector portion of a tube may be connected to the fluid entry opening 1015, which may enable one or more fluids to be transferred from a patient into the chest tube cannister 1000. The chest tube cannister 1000 may include a water seal chamber 1035 and/or an air leak monitor 1045. A suction port 1020 may enable air and/or one or more bodily fluids to be removed from the chest tube cannister 1000 via the water seal chamber 1035. The chest tube cannister 1000 may additionally or alternatively include a dry suction pressure regulator 1030, which may enable a user to manually control dry suction pressure within the chest tube cannister 1000.

The chest tube cannister 1000 of an example embodiment further includes one or more pressure sensors to monitor air flow into the cannister. Air flow monitoring of the chest tube cannister 1000 is beneficial in the case of a pneumothorax or collapsed lung. A collapsed lung occurs when air leaks into the space between a lung and the adjacent chest wall. The air pushes on the outside of the lung causing the collapse of the lung due to increasing pressure. A pneumothorax can be a total lung collapse or a partial lung collapse. In either scenario, it may be desirable to employ the chest tube to evacuate air in the pleural space between the lung and the chest wall. As air pressure decreases, the lung can re-expand and heal.

A tube inserted into the chest cavity that is associated with the chest tube cannister 1000 may be used to measure pressure inside of the chest to monitor pressure within the pleural space while treating the collapsed lung. The chest tube or chest tube cannister 1000 of an example embodiment can include pressure sensors anywhere along the path of airflow, such as in fluid entry opening 1015 and/or suction port 1020. Such pressure sensors can be used to monitor tube patency through pressure differentials to ensure the tube is therefore functioning properly and to monitor air evacuation from a patient's chest during treatment of a pneumothorax. The pressure sensors can be any conventional pressure sensor, such as an aneroid barometer or manometer pressure sensor which are mechanical sensors. However, in some circumstances, such as when the pressure sensors of the chest tube cannister 1000 are to be monitored by a controller, a sealed pressure sensor, piezoelectric pressure sensor, or strain gage type pressure sensor may be employed to provide an electronic signal to a controller for monitoring of the pressures seen at the chest tube cannister.

The chest tube cannister 1000 may include one or more buttons 605 and a display 610. In some examples, the chest tube cannister 1000 may include a battery chamber 1040 that includes one or more batteries for supplying power to one or more components of the chest tube cannister 1000, such as one or more processors, one or more communication components, the display 610, and/or the like. As described herein, the chest tube cannister 1000, or one or more components of the chest tube cannister 1000 may be an example of a fluid collection reservoir. For example, the one or more fluid collection chambers 1025 may be examples of one or more fluid collection reservoirs.

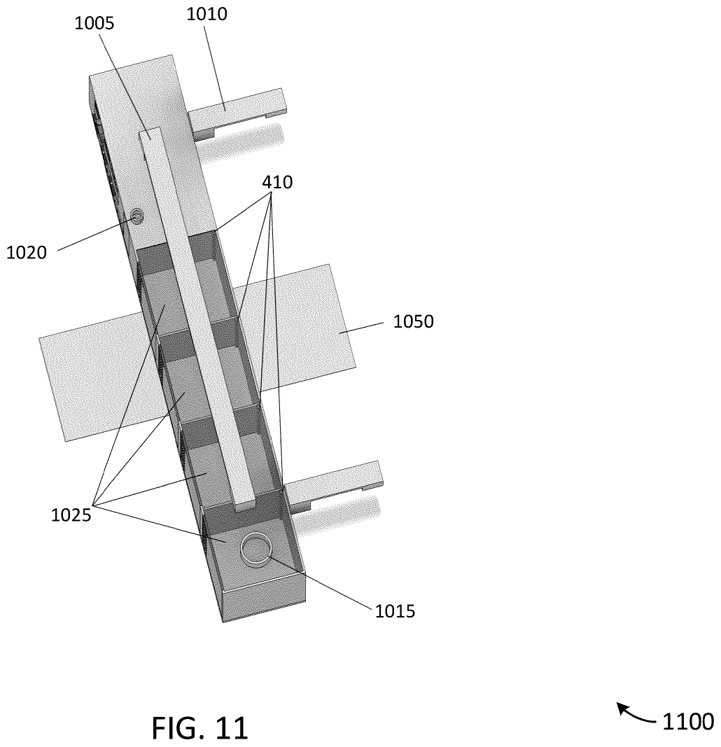

FIG. 11 illustrates an operational example of a chest tube cannister 1100 in accordance with some embodiments discussed herein. The chest tube cannister 1100 may be an example of the chest tube cannister 1000, described with reference to FIG. 10. As described herein, FIG. 11 may illustrate a top view of the chest tube cannister 1100. The chest tube cannister 1100 may be an example of a fluid measurement device configured to measure one or more bodily fluids, such as one or more fluids expelled by a patient during a medical procedure. The chest tube cannister 1100 may include one or more fluid collection chambers 1025, one or more capacitance sensors 410, one or more fluid entry openings 1015, one or more suction ports 1020, one or more handles 1005, one or more hangers 1010, one or more stabilizing components 1050, one or more point pressure sensors, one or more weight scale components, a base, one or more buttons, a display, an air leak monitor, a water seal chamber, a dry suction pressure regulator, and/or a battery chamber, which may be examples of corresponding elements described with reference to FIG. 10.

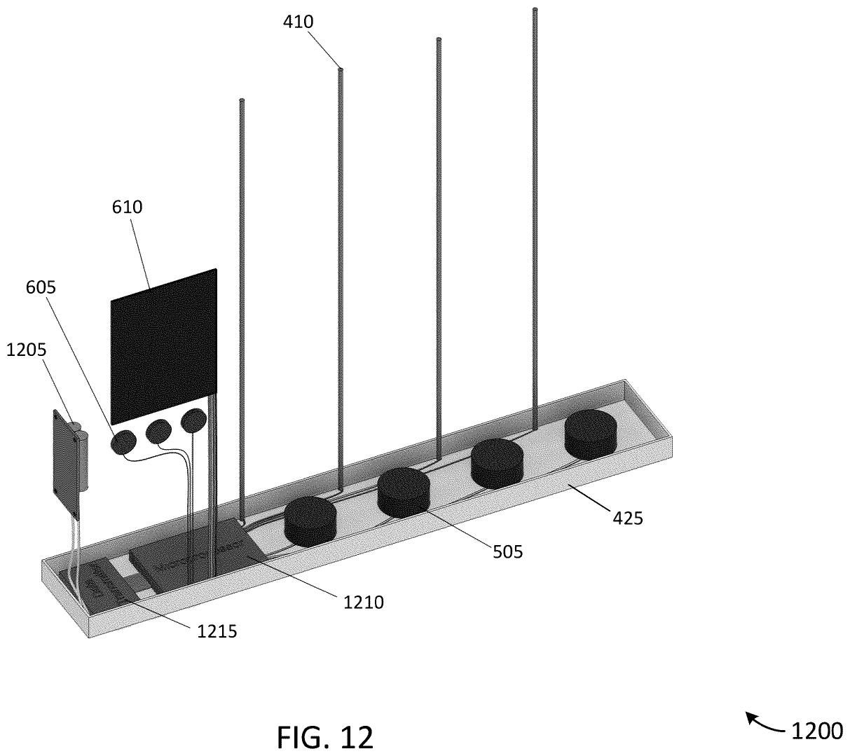

FIG. 12 illustrates an operational example of an electronics assembly 1200 in accordance with some embodiments discussed herein. The electronics assembly 1200 may include a base 425 of a fluid measurement device, which may house or be physically coupled with one or more other components of the electronics assembly 1200. The electronics assembly 1200 may include one or more processors 1210 (e.g., one or more microprocessors) and one or more communication interfaces 1215 (e.g., a data transmitter, a data receiver, a data transceiver) in communication with the one or more processors 1210. The one or more processors 1210 may communicate information, such as one or more fluid levels to the one or more communication interfaces 1215 wirelessly or via a wired connection. The one or more communication interfaces 1215 may then communicate the information to an electronic medical record and/or to one or more user devices (e.g., a phone, a smartwatch, a tablet, a computing entity). In some examples, the one or more processors 1210 may provide the information to a display 610, which may be viewable by a user, such as a medical professional and/or a patient. In such examples, the information may be communicated from the one or more processors 1210 to the display 610 via one or more wires, or any other type of electrical connection.

The electronics assembly 1200 may include one or more capacitance sensors 410 and/or one or more point pressure sensors 505, which may be electrically coupled with the one or more processors 1210 via one or more wires. Additionally, or alternatively, the electronics assembly 1200 may include one or more buttons 605, which may enable a user to control and/or provide information to the one or more processors 1210. For example, pressing a button 605 may cause the chest tube cannister (e.g., the one or more processors 1210) to power on, power off, display a battery life reading, and/or the like. Additionally, or alternatively, a user may control one or more functions of the chest tube cannister via the one or more buttons 605, such as selecting information displayed via the display 610, causing information to be stored or communicated, causing a measurement to be initiated, setting a clock, setting a timer for initiating one or more measurements, setting a data reporting interval for reporting data to a medical record, responding to one or more prompts, and/or the like. In some examples, the electronics assembly 1200 may include one or more batteries 1205, which may supply power to one or more components of the electronics assembly 1200 via one or more wires.

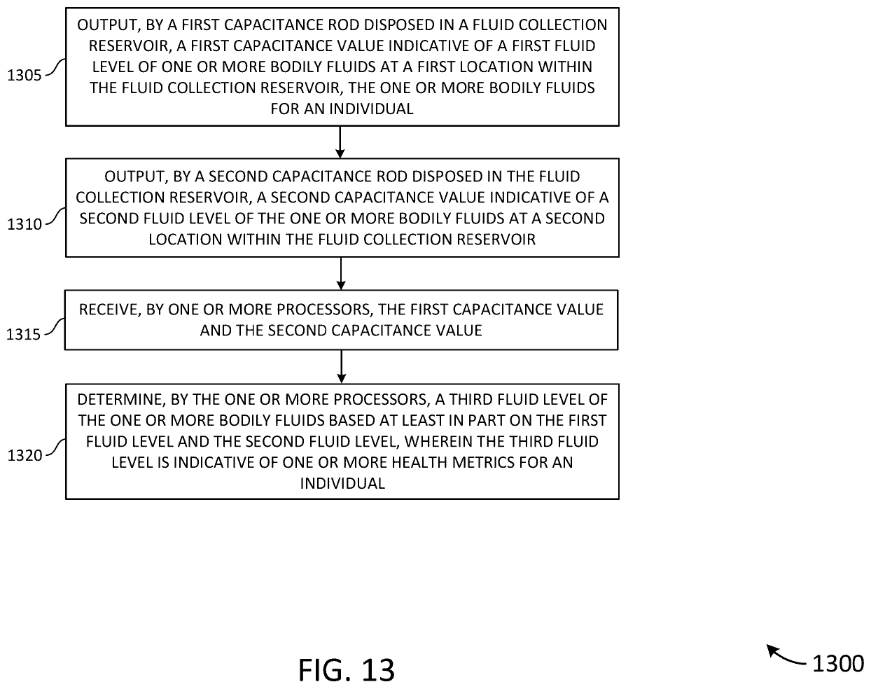

FIG. 13 provides an example flowchart 1300 in accordance with some embodiments described herein. The flowchart 1300 may include one or more examples of operations performed by a fluid measurement device or one or more subcomponents of a fluid measurement device, as described with reference to FIGS. 1-12. In accordance with examples described herein, any of the described operations may be performed multiple times or independently of other operations described herein.

As shown in block 1305, the fluid measurement device may include means, such as the one or more processors, the one or more capacitance sensors, and/or the like, for outputting, by a first capacitance sensor disposed in a fluid collection reservoir, a first capacitance value indicative of a first fluid level of one or more bodily fluids at a first location within the fluid collection reservoir, the one or more bodily fluids associated with an individual.

As shown in block 1310, the fluid measurement device may include means, such as the one or more processors, the one or more capacitance sensors, and/or the like, for outputting, by a second capacitance sensor disposed in the fluid collection reservoir, a second capacitance value indicative of a second fluid level of the one or more bodily fluids at a second location within the fluid collection reservoir.

As shown in block 1315, the fluid measurement device may include means, such as the one or more processors, the one or more capacitance sensors, and/or the like, for receiving, by one or more processors, the first capacitance value and the second capacitance value.

As shown in block 1320, the fluid measurement device may include means, such as the one or more processors, the one or more capacitance sensors, and/or the like, for determining, by the one or more processors, a third fluid level of the one or more bodily fluids based at least in part on the first fluid level and the second fluid level, wherein the third fluid level is indicative of one or more health metrics for the individual.

The flowchart 1300 depicts methods according to an example embodiment. It will be understood that each block and combination of blocks may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by memory of a computing entity as described herein. As will be appreciated, any such computer program instructions may be loaded onto a computer or other programmable apparatus (for example, hardware) to produce a machine, such that the resulting computer or other programmable apparatus implements the functions specified in the flowchart blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart blocks.

Accordingly, blocks of the flowcharts support combinations of means for performing the specified functions and combinations of operations for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

### VI. CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A system comprising:
a fluid collection reservoir configured to collect one or more bodily fluids associated with an individual;
a first capacitance sensor positioned in the fluid collection reservoir and configured to output a first capacitance value indicative of a first fluid level of the one or more bodily fluids at a first location within the fluid collection reservoir;
a second capacitance sensor positioned in the fluid collection reservoir and configured to output a second capacitance value indicative of a second fluid level of the one or more bodily fluids at a second location within the fluid collection reservoir; and
one or more processors in communication with the first capacitance sensor and the second capacitance sensor, the one or more processors configured to receive the first capacitance value and the second capacitance value and determine a third fluid level of the one or more bodily fluids based at least in part on the first fluid level and the second fluid level, wherein the third fluid level is indicative of one or more health metrics for the individual.

2. The system of claim 1, wherein the one or more processors are further configured to:
determine a tilt angle of the fluid collection reservoir based at least in part on a comparison of the first capacitance value and the second capacitance value, wherein the third fluid level is based at least in part on the tilt angle.

3. The system of claim 1, wherein the one or more processors are further configured to:
determine whether one or more measurement accuracy criteria are satisfied based at least in part on the first capacitance value and the second capacitance value; and
determine a measurement timing for determining the third fluid level based at least in part on whether the one or more measurement accuracy criteria are satisfied.

4. The system of claim 3, wherein the one or more measurement accuracy criteria comprise one or more of (i) a tilt level criteria and (ii) an agitation criteria.

5. The system of claim 1, wherein the one or more processors are further configured to:
generate a message comprising at least an indication of the third fluid level; and
cause transmission of the message to an electronic medical record for the individual.

6. The system of claim 5, wherein the one or more processors are further configured to receive an identification number for the individual and the electronic medical record for the individual is identified based at least in part on the identification number for the individual.

7. The system of claim 6, wherein the one or more processors configured to generate the message comprising at least the indication of the third fluid level comprise one or more processors configured to format the message according to a health level seven (HL7) messaging standard.

8. The system of claim 6, wherein the one or more processors configured to cause transmission of the message comprise one or more processors configured to encode the message according to a security protocol consistent with an electronic medical record system associated with the electronic medical record for the individual.

9. A method comprising:
outputting, by a first capacitance sensor disposed in a fluid collection reservoir, a first capacitance value indicative of a first fluid level of one or more bodily fluids at a first location within the fluid collection reservoir, the one or more bodily fluids associated with an individual;
outputting, by a second capacitance sensor disposed in the fluid collection reservoir, a second capacitance value indicative of a second fluid level of the one or more bodily fluids at a second location within the fluid collection reservoir;
receiving, by one or more processors, the first capacitance value and the second capacitance value; and
determining, by the one or more processors, a third fluid level of the one or more bodily fluids based at least in part on the first fluid level and the second fluid level, wherein the third fluid level is indicative of one or more health metrics for the individual.

10. The method of claim 9, further comprising:
determining, by the one or more processors, a tilt angle of the fluid collection reservoir based at least in part on a comparison of the first capacitance value and the second capacitance value, wherein the third fluid level is based at least in part on the tilt angle.

11. The method of claim 9, further comprising:
determining, by the one or more processors, whether one or more measurement accuracy criteria are satisfied based at least in part on the first capacitance value and the second capacitance value; and
determining, by the one or more processors, a measurement timing for determining the third fluid level based at least in part on whether the one or more measurement accuracy criteria are satisfied.

12. The method of claim 11, wherein the one or more measurement accuracy criteria comprise one or more of (i) a tilt level criteria and (ii) an agitation criteria.

13. The method of claim 9, further comprising:
generating, by the one or more processors, a message comprising at least an indication of the third fluid level; and
causing, by the one or more processors, transmission of the message to an electronic medical record for the individual.

14. The method of claim 13, further comprising:
receiving, by the one or more processors, an identification number for the individual, wherein the electronic medical record for the individual is identified based at least in part on the identification number for the individual.

15. The method of claim 14, further comprising:
formatting, by the one or more processors, the message according to a health level seven (HL7) messaging standard.

16. The method of claim 14, further comprising:
encoding, by the one or more processors, the message according to a security protocol consistent with an electronic medical record system associated with the electronic medical record for the individual.

17. An apparatus comprising:
at least one processor; and
at least one memory storing instructions that, when executed by the at least one processor, cause the apparatus to:
output, by a first capacitance sensor disposed in a fluid collection reservoir, a first capacitance value indicative of a first fluid level of one or more bodily fluids at a first location within the fluid collection reservoir, the one or more bodily fluids associated with an individual;
output, by a second capacitance sensor disposed in the fluid collection reservoir, a second capacitance value indicative of a second fluid level of the one or more bodily fluids at a second location within the fluid collection reservoir;
receive, by the at least one processor, the first capacitance value and the second capacitance value; and
determine, by the at least one processor, a third fluid level of the one or more bodily fluids based at least in part on the first fluid level and the second fluid level, wherein the third fluid level is indicative of one or more health metrics for the individual.

18. The apparatus of claim 17, wherein the instructions, when executed by the at least one processor further cause the apparatus to:
determine, by the at least one processor, a tilt angle of the fluid collection reservoir based at least in part on a comparison of the first capacitance value and the second capacitance value, wherein the third fluid level is based at least in part on the tilt angle.

19. The apparatus of claim 17, wherein the instructions, when executed by the at least one processor further cause the apparatus to:
determine, by the at least one processor, whether one or more measurement accuracy criteria are satisfied based at least in part on the first capacitance value and the second capacitance value; and
determine, by the at least one processor, a measurement timing for determining the third fluid level based at least in part on whether the one or more measurement accuracy criteria are satisfied.

20. The apparatus of claim 17, wherein the instructions, when executed by the at least one processor further cause the apparatus to:
generate, by the at least one processor, a message comprising at least an indication of the third fluid level; and
cause, by the at least one processor, transmission of the message to an electronic medical record for the individual.
